(19) United States
(12) Patent Application Publication
(10) Pub. No.: US 2025/0388972 A1
(43) Pub. Date: Dec. 25, 2025

(54) COMPOSITIONS AND METHODS FOR DETECTION OF METASTASIS

(71) Applicant: GUARDANT HEALTH, INC., Palo Alto, CA (US)

(72) Inventors: AmirAli TALASAZ, Atherton, CA (US); Andrew KENNEDY, San Diego, CA (US); Scott R. BORTNER, Palo Alto, CA (US)

(21) Appl. No.: 18/750,440

(22) Filed: Jun. 21, 2024

Related U.S. Application Data
(63) Continuation of application No. PCT/US2022/082257, filed on Dec. 22, 2022.
(60) Provisional application No. 63/293,524, filed on Dec. 23, 2021.

(51) Int. Cl. C12Q 1/6886; C12Q 1/6869; G01N 33/68

(52) U.S. Cl. CPC C12Q 1/6886; C12Q 1/6869; G01N 33/6893; C12Q 2600/154; G01N 2333/70521; G01N 2333/70532

(57) ABSTRACT

Provided herein are methods of detecting the presence or absence of metastasis in a subject comprising detecting one or more cell materials released from a potential metastasis site. In some embodiments, the one or more cell materials detected from the potential metastasis site are released from otherwise healthy cells due to shedding or cell death induced by a metastasis.

### CROSS-REFERENCE TO RELATED APPLICATIONS

This application is a continuation of International PCT Application No. PCT/US2022/082257, filed on Dec. 22, 2022, which claims the benefit of U.S. Provisional Application No. 63/293,524, filed on Dec. 23, 2021, both of which are incorporated herein by reference for all purposes.

### FIELD OF THE INVENTION

The present disclosure provides compositions and methods related to detecting metastasis in a subject. In some embodiments, the methods comprise detecting cell materials released from a metastasis site. In some embodiments, the cell materials released from the metastasis site are from otherwise healthy cells or tissues that were invaded by the metastasis. In some embodiments, the detection of the cell materials facilitates determination of the likelihood that the subject has a metastasis.

### INTRODUCTION AND SUMMARY

Cancer is responsible for millions of deaths per year worldwide. Improperly controlled cell growth is a hallmark of cancer that generally results from an accumulation of genetic and epigenetic changes, such as copy number variations (CNVs), single nucleotide variations (SNVs), gene fusions, insertions and/or deletions (indels), epigenetic variations including modification of cytosine (e.g., 5-methylcytosine, 5-hydroxymethylcytosine, and other more oxidized forms) and association of DNA with chromatin proteins and transcription factors.

As cancer progresses, it may metastasize to tissues distal to the site of the primary cancer or tumor. Detection of metastasis is important in order to monitor cancer progression and to adjust treatment as needed. Both the presence of a metastasis and the location or locations of the metastasis or metastases are critical to treatment selections. Current detection methods for metastasis include a mixture of imaging and detection of markers associated with the primary cancer or tumor. There is a need for streamlined, direct methods for detecting metastasis.

Detection of cancer based on analysis of body fluids (“liquid biopsies”), such as blood, is a non-invasive method based on the observation that biomolecule materials from cancer cells is released into body fluids. Such non-invasive detection methods may be adapted for detection of metastases.

Without wishing to be bound by any particular theory, cells in or around a metastatic cancer or neoplasm, such as a tissue invaded by a metastasis, may shed more DNA, cell debris, and other cell materials than cells of the same tissue type that are not invaded by a metastasis. As such, the presence or level of cell material from apparently healthy tissues may change upon metastatic invasion of the tissue. Thus, for example, in a sample from a subject having a primary cancer of a first tissue type, an increase in the level of methylated DNA corresponding to a second tissue type different from the first tissue type, relative to the level thereof present in the absence of metastasis, can be an indicator of the presence of a metastasis at the site of the second tissue.

Methods according to this disclosure may provide information about whether a metastasis is present and the tissue type of the metastasis site based on one or more blood samples obtained from a subject. The methods may further provide combined information about cell material associated with the primary cancer or tumor and cell material associated with the tissue of the metastasis type.

The present disclosure aims to meet the need for improved detection of metastasis. Accordingly, the following exemplary embodiments are provided.
Embodiment 1. A method of detecting the presence or absence of a metastasis in a subject, the method comprising:
detecting a presence or level of at least one cell material released from a potential metastasis site in a sample from the subject;
detecting the presence or absence of the metastasis based at least in part on the presence or level of the at least one cell material released from the potential metastasis site;
wherein the at least one cell material released from the potential metastasis site comprises one or more of tissue-specific hydroxymethylated DNA; tissue-specific fragmented DNA; a tissue-specific modified histone; a tissue-specific bacterial nucleic acid; a tissue-specific protein or cell debris; a tissue-specific extracellular vesicle; or a tissue-specific RNA.
Embodiment 2. A method of detecting the presence or absence of a metastasis in a subject, the method comprising:
detecting a presence or level of at least one cell material released from a potential metastasis site in a sample from the subject;
detecting whether the presence or level of the at least one cell material released from the potential metastasis site has changed relative to the presence or level of the at least one cell material in a previous sample from the subject, wherein the previous sample was obtained at an earlier time than the sample;
detecting the presence or absence of the metastasis based at least in part on a change, or lack thereof, in the presence or level of the at least one cell material released from the potential metastasis site between the previous sample and the sample.
Embodiment 3. The method of embodiment 2, wherein the previous sample was obtained before the subject underwent a cancer treatment, and the sample was obtained after the treatment; or the previous sample was obtained within one month of the subject receiving a cancer diagnosis; or the previous sample was obtained at least 3, 6, 9, 12, 18, or 24 months before the sample.
Embodiment 4. A method of detecting the presence or absence of a metastasis in a subject, the method comprising:
detecting a relative level of at least one cell material released from a potential metastasis site in a sample from the subject;
detecting the presence or absence of the metastasis based at least in part on the relative level of the at least one cell material released from the potential metastasis site; wherein the relative level of the at least one cell material is the level of the at least one cell material relative to the level of a comparator cell material.
Embodiment 5. The method of embodiment 2 or 3, wherein the presence or level of the at least one cell material released from a potential metastasis site is a relative level, wherein the relative level of the at least one cell material is the level of the at least one cell material relative to the level of a comparator cell material, and wherein the detecting the presence or absence of the metastasis based at least in part on a change, or lack thereof, in the relative level of the at least one cell material released from the potential metastasis site between the previous sample and the sample.
Embodiment 6. The method of embodiment 4 or 5, wherein the comparator cell material comprises cell material released from one or more cell or tissue types selected from erythroid cell or tissue, healthy cell or tissue, and primary cancer, tumor, or neoplastic cell.
Embodiment 7. The method of any one of embodiments 4-6, wherein the comparator cell material comprises a heterologous comparator cell material.
Embodiment 8. The method of the immediately preceding embodiment, wherein the heterologous comparator cell material is cell material obtained from a reference population of subjects.
Embodiment 9. The method of any one of embodiments 4-8, wherein the level of the comparator cell material is determined from samples obtained from a reference population of healthy subjects.
Embodiment 10. The method of any one of embodiments 4-6, wherein the comparator cell material comprises an autologous comparator cell material.
Embodiment 11. The method of the immediately preceding embodiment, wherein the autologous cell material is cell material obtained from the subject when the subject did not have a metastasis or did not have a primary cancer, tumor, or neoplasm.
Embodiment 12. The method of any one of embodiments 4-6, 10, or 11, wherein the level of the comparator cell material is determined from one or more samples obtained from the subject when the subject did not have a metastasis or did not have a primary cancer, tumor, or neoplasm.
Embodiment 13. The method of any one of embodiments 2-12, wherein the at least one cell material released from the potential metastasis site comprises tissue-specific methylated DNA.
Embodiment 14. The method of any one of embodiments 2-12, wherein the at least one cell material released from the potential metastasis site comprises one or more of tissue-specific hydroxymethylated DNA; tissue-specific fragmented DNA; a tissue-specific modified histone; a tissue-specific bacterial nucleic acid; a tissue-specific protein or cell debris; or a tissue-specific RNA.
Embodiment 15. The method of any one of the preceding embodiments, wherein the at least one cell material released from the potential metastasis site comprises cell-free cell material.
Embodiment 16. The method of any one of the preceding embodiments, wherein the least one cell material released from the potential metastasis site comprises a plurality of cell materials released from the potential metastasis site.
Embodiment 17. The method of any one of the preceding embodiments, wherein at least one cell material released from the potential metastasis site comprises tissue-specific bacterial nucleic acid.
Embodiment 18. The method of the immediately preceding embodiment, wherein the tissue-specific bacterial nucleic acid is released from bacteria located in the gut, mouth, or reproductive organs.
Embodiment 19. The method of embodiment 17 or 18, wherein the tissue-specific bacterial nucleic acid is released from Bilophila wadsworthia, Streptococcus bovis, Helicobacter pylori, Bacteroides fragilis, or Clostridium septicum.
Embodiment 20. The method of any of one of embodiments 17-19, wherein the tissue-specific bacterial nucleic acid is a cell-free nucleic acid.
Embodiment 21. The method of any one of embodiments 17-20, wherein the tissue-specific bacterial nucleic acid is a 16S rRNA or DNA encoding a 16S rRNA.
Embodiment 22. The method of any one of the preceding embodiments, wherein at least one cell material released from the potential metastasis site comprises a tissue-specific epigenetic target region of a nucleic acid.
Embodiment 23. The method of the immediately preceding embodiment, wherein the tissue-specific epigenetic target region comprises a region of tissue-specific methylated DNA.
Embodiment 24. The method of the immediately preceding embodiment, wherein the tissue-specific methylated DNA comprises cfDNA.
Embodiment 25. The method of any one of embodiments 13, 23, or 24, wherein the tissue-specific methylated DNA is specific to the brain, lung, skin, nose, throat, liver, bone, bone marrow, pancreas, lymph nodes, bowel, rectum, colon, prostate, thyroid, bladder, head, neck, kidney, mouth, stomach, or spleen.
Embodiment 26. The method of any one of embodiments 13, 23, or 24, wherein the tissue-specific methylated DNA is specific to the colon, lymph nodes, brain, liver, or spleen.
Embodiment 27. The method of any one of the preceding embodiments, wherein at least one cell material released from the potential metastasis site comprises a tissue-specific RNA selected from a tissue-specific microRNA, a tissue-specific exosomal RNA, or a tissue-specific extracellular RNA.
Embodiment 28. The method of any one of the preceding embodiments, where at least one cell material released from the potential metastasis site comprises tissue-specific cell debris comprising a protein, carbohydrate, and/or cell debris marker.
Embodiment 29. The method of the immediately preceding embodiment, wherein the tissue-specific cell debris comprises PD-L1, CTLA4, NYESO1, mesothelin, CA15-3, CA19-9, CA-125, or CA-172-4.
Embodiment 30. The method of any one of the preceding embodiments, wherein the primary cancer, tumor, or neoplasm is a hematological cancer.
Embodiment 31. The method of the immediately preceding embodiment, wherein the hematological cancer is a lymphoma, a leukemia, or multiple myeloma.
Embodiment 32. The method of any one of embodiments 1-29, wherein the primary cancer, tumor, or neoplasm is a cancer, tumor, or neoplasm of the liver, skin, lung, breast, or pancreas.
Embodiment 33. The method of any one of embodiments 1-29, wherein the subject has a cancer of unknown primary.
Embodiment 34. The method of any one of the preceding embodiments, wherein the potential metastasis site is the brain, lung, skin, nose, throat, liver, bone, bone marrow, pancreas, lymph nodes, bowel, rectum, colon, prostate, thyroid, bladder, head, neck, kidney, mouth, stomach, or spleen.
Embodiment 35. The method of any one of the preceding embodiments, wherein the potential metastasis site is the colon, lymph nodes, brain, liver, or spleen.
Embodiment 36. The method of any one of the preceding embodiments, wherein the presence of the metastasis is detected based at least in part on detection of a level of at least one cell material released from the metastasis site that is higher than expected for healthy tissue that has not been invaded by a metastasis.
Embodiment 37. The method of the immediately preceding embodiment, wherein the higher than expected level of at least one cell material released from the metastasis site is higher relative to the level detected in a previous sample or relative to a comparator cell material.
Embodiment 38. The method of any one of the preceding embodiments, further comprising imaging the potential metastasis site.
Embodiment 39. The method of the immediately preceding embodiment, wherein the imaging is performed after the detecting of the presence or level of the at least one cell material released from the metastasis site.
Embodiment 40. The method of any one of the preceding embodiments, wherein the sample is a blood sample.
Embodiment 41. The method of the immediately preceding embodiment, wherein the blood sample is a whole blood sample.
Embodiment 42. The method of any one of embodiments 1-40, wherein the sample comprises plasma obtained from a blood sample.
Embodiment 43. The method of any one of embodiments 1-40, wherein the sample comprises serum.
Embodiment 44. The method of any one of embodiments 1-39, wherein the sample is a tissue sample.
Embodiment 45. The method of the immediately preceding embodiment, wherein the tissue sample is a biopsy, a fine needle aspirate, or a formalin-fixed paraffin-embedded tissue sample.
Embodiment 46. The method of any one the preceding embodiments, wherein the sample comprises cfDNA.
Embodiment 47. The method of any one of the preceding embodiments, further comprising detecting the primary cancer, tumor, or neoplasm based at least in part on detecting at least one cell material released from one or more primary cancer, tumor, or neoplasm cells or from tissue in which the primary cancer, tumor, or neoplasm cells are located.
Embodiment 48. The method of the immediately preceding embodiment, wherein the detecting at least one cell material released from the primary cancer, tumor, or neoplasm comprises capturing a plurality of sets of target regions of DNA from the sample or one or more subsamples thereof, wherein the plurality of sets of target regions comprises a sequence-variable target region set and an epigenetic target region set, thereby providing captured DNA.
Embodiment 49. The method of the immediately preceding embodiment, comprising sequencing the captured DNA.
Embodiment 50. The method of any one of the preceding embodiments, wherein the subject was diagnosed with cancer before the sample was obtained.
Embodiment 51. The method of any one of the preceding embodiments, wherein the subject received treatment for a cancer before the sample was obtained.
Embodiment 52. The method of any one of the preceding embodiments, wherein the subject is undergoing screening for cancer.
Embodiment 53. The method of any one of the preceding embodiments, wherein the subject has a metastasis, and the method further comprises identifying the metastasis site based at least in part on the at least one cell material.
Embodiment 54. The method of the immediately preceding embodiment, wherein the metastasis site is the brain, lung, skin, nose, throat, liver, bone, bone marrow, pancreas, lymph nodes, bowel, rectum, colon, prostate, thyroid, bladder, head, neck, kidney, mouth, stomach, or spleen
Embodiment 55. The method of any one of the preceding embodiments, further comprising detecting organ failure at a metastasis site.
Embodiment 56. The method of any one of the preceding embodiments, further comprising detecting one or more metastasis-associated sequence variants.
Embodiment 57. The method of any one of the preceding embodiments, wherein the detecting the presence, level, or relative level of the at least one cell material from the potential metastasis site comprises partitioning the sample into a plurality of subsamples by contacting the sample with an agent that recognizes a cell debris marker, a chromatin-associated target, or a nucleic acid modification, the plurality comprising a first subsample and a second subsample, wherein the first subsample comprises the cell debris marker, chromatin-associated target, or nucleic acid modification in a greater proportion than the second subsample.
Embodiment 58. The method of the immediately preceding embodiment, wherein the agent recognizes a nucleic acid modification, wherein the nucleic acid modification is methylated cytosine in DNA.
Embodiment 59. The method of embodiment 57 or 58, wherein the partitioning the sample into a plurality of subsamples comprises partitioning on the basis of methylation level of nucleic acids.
Embodiment 60. The method of the immediately preceding embodiment, wherein the agent recognizes a nucleic acid modification is a methyl binding reagent.
Embodiment 61. The method of the immediately preceding embodiment, wherein the methyl binding reagent specifically recognizes 5-methylcytosine.
Embodiment 62. The method of any one of embodiments 57-61, wherein the agent is immobilized on a solid support.
Embodiment 63. The method of any one of embodiments 57-62, wherein partitioning the sample into a plurality of subsamples comprises immunoprecipitation of the cell material bound to the agent.
Embodiment 64. The method of any one of embodiments 57-63, comprising differentially tagging and pooling DNA of the first subsample and second subsample.
Embodiment 65. The method of any one of embodiments 57-64, wherein the DNA of the first subsample and the DNA of the second subsample are differentially tagged; after differential tagging, a portion of DNA from the second subsample is added to the first subsample or at least a portion thereof, thereby forming a pool; and sequence-variable target regions and epigenetic target regions are captured from the pool.
Embodiment 66. The method of the immediately preceding embodiment, wherein the pool comprises less than or equal to about 45%, 40%, 35%, 30%, 25%, 20%, 15%, 10%, or 5% of the DNA of the second subsample.
Embodiment 67. The method of the immediately preceding embodiment, wherein the pool comprises about 70-90%, about 75-85%, or about 80% of the DNA of the second subsample.
Embodiment 68. The method of any one of embodiments 65-67, wherein the pool comprises substantially all of the DNA of the first subsample.
Embodiment 69. The method of any one of embodiments 58-68, wherein the plurality of subsamples comprises a third subsample, which comprises DNA with a cytosine modification in a greater proportion than the second subsample but in a lesser proportion than the first subsample.
Embodiment 70. The method of the immediately preceding embodiment, wherein the method further comprises differentially tagging the third subsample.
Embodiment 71. The method of any one of the preceding embodiments, wherein the detecting the presence, level, or relative level of the at least one cell material from the potential metastasis site comprises subjecting the sample or a subsample thereof to a procedure that affects a first nucleobase in DNA differently from a second nucleobase in DNA, wherein the first nucleobase is a modified or unmodified nucleobase, the second nucleobase is a modified or unmodified nucleobase different from the first nucleobase, and the first nucleobase and the second nucleobase have the same base pairing specificity.
Embodiment 72. The method of the immediately preceding embodiment, wherein the procedure to which the sample or a subsample thereof is subjected alters base-pairing specificity of the first nucleobase without substantially altering base-pairing specificity of the second nucleobase.
Embodiment 73. The method of embodiment 71 or 72, wherein the first nucleobase is a modified or unmodified cytosine, and the second nucleobase is a modified or unmodified cytosine.
Embodiment 74. The method of any one of embodiments 71-73, wherein the first nucleobase comprises unmodified cytosine or 5-methylcytosine (5mC).
Embodiment 75. The method of any one of embodiments 71-73, wherein the second nucleobase comprises 5mC or 5-hydroxymethylcytosine (5hmC).
Embodiment 76. The method of any one of embodiments 71-75, wherein the procedure to which the sample or a subsample thereof is subjected comprises bisulfite conversion.
Embodiment 77. The method of any one of the preceding embodiments, wherein the detecting the presence, level, or relative level of the at least one cell material from the potential metastasis site comprises detecting nucleic acids obtained from the sample or a subsample thereof.
Embodiment 78. The method of the immediately preceding embodiment, wherein the detecting nucleic acids obtained from the sample or a subsample thereof comprises sequencing nucleic acids obtained from the sample or a subsample thereof.
Embodiment 79. The method of embodiment 77, wherein the detecting nucleic acids obtained from the sample or a subsample thereof comprises amplifying nucleic acids obtained from the sample or a subsample thereof by quantitative or digital PCR.
Embodiment 80. The method of any one of embodiments 49-78, wherein the sequencing comprises high-throughput sequencing, pyrosequencing, sequencing-by-synthesis, single-molecule sequencing, nanopore-based sequencing, semiconductor sequencing, sequencing-by-ligation, sequencing-by-hybridization, RNA-Seq (Illumina), Digital Gene Expression (Helicos), next generation sequencing (NGS), Single Molecule Sequencing by Synthesis (SMSS) (Helicos), massively-parallel sequencing, Clonal Single Molecule Array (Solexa), shotgun sequencing, Ion Torrent, Oxford Nanopore, Roche Genia, Maxim-Gilbert sequencing, primer walking, sequencing using PacBio, SOLiD, Ion Torrent, or a Nanopore platform.
Embodiment 81. The method of any one of the preceding embodiments, wherein the subject is a human.
Embodiment 82. The method of any one of embodiments 22-81, wherein the epigenetic target regions comprise a hypermethylation variable target region set.
Embodiment 83. The method of any one of embodiments 22-82, wherein the epigenetic target regions comprise a fragmentation variable target region set.
Embodiment 84. The method of the immediately preceding embodiment, wherein the fragmentation variable target region set comprises at least one of transcription start site regions or CTCF binding regions.
Embodiment 85. The method of any one of the preceding embodiments, comprising determining a cancer recurrence or metastatic score that is indicative of the presence or absence of recurrence or of a metastasis, wherein the presence of recurrence or of metastasis in the subject is determined to be likely when the recurrence or metastatic score is determined to be at or above a predetermined threshold, or the presence of recurrence or of metastasis in the subject is determined to be unlikely when the recurrence or metastatic score is below the predetermined threshold.
Embodiment 86. The method of the immediately preceding embodiment, further comprising comparing the recurrence or metastatic score of the subject with a predetermined threshold, wherein the subject is classified as a candidate for a certain cancer treatment when the recurrence or metastatic score is above the threshold or not a candidate for the certain cancer treatment when the recurrence or metastatic score is below the threshold.

### BRIEF DESCRIPTION OF THE DRAWINGS

FIG. 1 is a schematic diagram of an example of a system suitable for use with some embodiments of the disclosure.

### DETAILED DESCRIPTION OF CERTAIN EMBODIMENTS

Reference will now be made in detail to certain embodiments of the invention. While the invention will be described in conjunction with such embodiments, it will be understood that they are not intended to limit the invention to those embodiments. On the contrary, the invention is intended to cover all alternatives, modifications, and equivalents, which may be included within the invention as defined by the appended claims.

Before describing the present teachings in detail, it is to be understood that the disclosure is not limited to specific compositions or process steps, as such may vary. It should be noted that, as used in this specification and the appended claims, the singular form “a”, “an” and “the” include plural references unless the context clearly dictates otherwise. Thus, for example, reference to “a nucleic acid” includes a plurality of nucleic acids, reference to “a cell” includes a plurality of cells, and the like.

Numeric ranges are inclusive of the numbers defining the range. Measured and measurable values are understood to be approximate, taking into account significant digits and the error associated with the measurement. Also, the use of “comprise”, “comprises”, “comprising”, “contain”, “contains”, “containing”, “include”, “includes”, and “including” are not intended to be limiting. It is to be understood that both the foregoing general description and detailed description are exemplary and explanatory only and are not restrictive of the teachings.

Unless specifically noted in the specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; and embodiments in the specification that recite “consisting essentially of” various components are also contemplated as “consisting of” or “comprising” the recited components (this interchangeability does not apply to the use of these terms in the claims).

The section headings used herein are for organizational purposes and are not to be construed as limiting the disclosed subject matter in any way. In the event that any document or other material incorporated by reference contradicts any explicit content of this specification, including definitions, this specification controls.

### I. Definitions

“Cell-free DNA,” “cfDNA molecules,” or simply “cfDNA” include DNA molecules that naturally occur in a subject in extracellular form (e.g., in blood, serum, plasma, or other bodily fluids such as lymph, cerebrospinal fluid, urine, or sputum). While the cfDNA previously existed in a cell or cells in a large complex biological organism, e.g., a mammal, it has undergone release from the cell(s) into a fluid found in the organism, and may be obtained from a sample of the fluid without the need to perform an in vitro cell lysis step. cfDNA molecules may occur as DNA fragments. cfDNA may be free of histones or nucleosomes, or cfDNA may be associated with histones or nucleosomes and thus part of chromatin fragments.

“Cell debris” as used herein means components of dead cells that can be released into the blood or other bodily fluids following apoptosis, autophagic cell death, necrosis, or other types of cell death. For example, cell death can lead to fragmentation of cell membranes into biomolecular complexes containing cell surface proteins. In some embodiments, cell debris comprises membrane fragments released from a dead or dying cell and associated molecules such as proteins and/or carbohydrates. Cell debris excludes nucleic acids.

“Cell debris marker” as used herein means a molecule, such as a protein, lipid, or carbohydrate, that is physically associated with or embedded in a component of a dead or a dying cell and is present in greater proportion in such components of ruptured or intact dead or dying cells than on the outer membrane of intact live cells, intact vesicles, or in the soluble fraction of a sample. The component of the dead or dying cell associated with the cell debris marker may be dissociated from other components of the cell from which it originated or may be contained in an intact dead or dying cell. Examples of cell debris markers include but are not limited to molecules associated with or localized to the inner plasma membrane, e.g., phosphatidylserine and phosphatidylethanolamine.

As used herein, “chromatin-associated targets” mean molecules, such as proteins, that are part of or bind directly or indirectly to chromatin. Chromatin-associated targets needs not be associated with chromatin at all times. Nucleosome-associated targets are a subset of chromatin-associated targets that are part of or bind directly or indirectly to nucleosomes and include histones. Agents that bind to chromatin-associated targets may be specific for an unmodified or modified form of the target.

“Cell material” as used herein means molecules made by one or more cells or components of one or more cells. “Cell-free cell material” is naturally extracellular cell material. While the cell-free cell material previously existed as part of a cell or cells in a large complex biological organism, e.g., a mammal, it has undergone secretion or release from the cell(s) into a fluid found in the organism, and may be obtained from a sample of the fluid without the need to perform an in vitro cell lysis step. Cell material, such as cell-free cell material, can be released into the blood or other bodily fluids, secreted, or released from one or more live cells, and/or released following apoptosis, autophagic cell death, necrosis, or other types of cell death. Examples of cell materials include but are not limited to DNA, including cfDNA, RNA, chromatin, histones, proteins, membrane fragments and other lipids, and vesicles and exosomes. In some embodiments, levels of different cell materials are compared in order to determine a relative level of a cell material. A “comparator cell material” is a cell material, the level of which has been or can be determined and is compared to the level of another cell material. Such a comparison can be used to determine a relative level of a cell material.

“Metastasis site” as used herein means a tissue or organ in an individual having a tumor, cancer, or neoplasm to which the tumor, cancer, or neoplasm has spread or metastasized. A metastasis site is a different tissue or organ than the tissue or organ that gave rise to the primary tumor, cancer, or neoplasm. A “potential metastasis site” is a tissue or organ in an individual having a tumor, cancer, or neoplasm located elsewhere to which the tumor, cancer, or neoplasm may spread or metastasize, or may have already spread or metastasized. A potential metastasis site may or may not be an actual metastasis site. Some embodiments of the methods herein can be used to determine whether or not a potential metastasis site is an actual metastasis site. In some embodiments, the tumor, cancer, or neoplasm that has spread or metastasized is a primary tumor, cancer, or neoplasm.

As used herein, a “metastasis-associated sequence variant” is one or more nucleic acid mutations that promote or are correlated with metastasis by a primary cancer.

As used herein, “cancer of unknown primary” means a cancer of a type that has not been identified.

As used herein, “partitioning” of a sample, means separating, fractionating, or sorting a sample into a plurality of subsamples based on one or more modifications or features of material that is present in different proportions in each of the plurality of subsamples or subpopulations. Partitioning may include physically partitioning cell materials based on the presence or absence of one or more lipids, post-translational protein modifications, or methylated nucleobases in nucleic acids, such as DNA. A sample may be partitioned into a plurality of partitioned subsamples based on a modification or feature that is indicative of a genetic or epigenetic change, a disease state, or one or more specific tissue types.

As used herein, “enriching” or “capturing” one or more molecules or complexes of interest refers to isolating or separating the one or more molecules or complexes of interest from other molecules or complexes. Partitioning is a type of enrichment in which molecules are separated into a plurality of subsamples.

As used herein, a modification or other feature is present in “a greater proportion” in a first sample or subsample than in second sample or subsample when the fraction of molecules with the modification or other feature is higher in the first sample or subsample than in the second sample or subsample. For example, if in a first sample comprising DNA, one tenth of the nucleotides are 5mC, and in a second sample comprising DNA, one twentieth of the nucleotides are 5mC, then the first sample comprises the cytosine modification of 5-methylation in a greater proportion than the second sample.

As used herein, the form of the “originally isolated” sample refers to the composition or chemical structure of a sample at the time it was isolated and before undergoing any procedure that changes the chemical structure of biomolecules or cell material in the isolated sample. Similarly, a feature that is “originally present” in cell material refers to a feature present in cell material “originally comprising” the feature before it undergoes any procedure that changes the chemical structure of the cell material.

As used herein, “without substantially altering base pairing specificity” of a given nucleobase means that a majority of molecules comprising that nucleobase that can be sequenced do not have alterations of the base pairing specificity of the given nucleobase relative to its base pairing specificity as it was in the originally isolated sample. In some embodiments, 75%, 90%, 95%, or 99% of molecules comprising that nucleobase that can be sequenced do not have alterations of the base pairing specificity relative to its base pairing specificity as it was in the originally isolated sample. As used herein, “altered base pairing specificity” of a given nucleobase means that a majority of molecules comprising that nucleobase that can be sequenced have a base pairing specificity at that nucleobase relative to its base pairing specificity in the originally isolated sample.

As used herein, “base pairing specificity” refers to the standard DNA base (A, C, G, or T) for which a given base most preferentially pairs. For example, unmodified cytosine and 5-methylcytosine have the same base pairing specificity (i.e., specificity for G) whereas uracil and cytosine have different base pairing specificity because uracil has base pairing specificity for A while cytosine has base pairing specificity for G. The ability of uracil to form a wobble pair with G is irrelevant because uracil nonetheless most preferentially pairs with A among the four standard DNA bases.

As used herein, a “combination” comprising a plurality of members refers to either of a single composition comprising the members or a set of compositions in proximity, e.g., in separate containers or compartments within a larger container, such as a multiwell plate, tube rack, refrigerator, freezer, incubator, water bath, ice bucket, machine, or other form of storage.

The “capture yield” of a collection of probes for a given target set refers to the amount (e.g., amount relative to another target set or an absolute amount) of nucleic acid corresponding to the target set that the collection of probes captures under typical conditions. Exemplary typical capture conditions are an incubation of the sample nucleic acid and probes at 65° C. for 10-18 hours in a small reaction volume (about 20 μL) containing stringent hybridization buffer. The capture yield may be expressed in absolute terms or, for a plurality of collections of probes, relative terms. When capture yields for a plurality of sets of target regions are compared, they are normalized for the footprint size of the target region set (e.g., on a per-kilobase basis). Thus, for example, if the footprint sizes of first and second target regions are 50 kb and 500 kb, respectively (giving a normalization factor of 0.1), then the DNA corresponding to the first target region set is captured with a higher yield than DNA corresponding to the second target region set when the mass per volume concentration of the captured DNA corresponding to the first target region set is more than 0.1 times the mass per volume concentration of the captured DNA corresponding to the second target region set. As a further example, using the same footprint sizes, if the captured DNA corresponding to the first target region set has a mass per volume concentration of 0.2 times the mass per volume concentration of the captured DNA corresponding to the second target region set, then the DNA corresponding to the first target region set was captured with a two-fold greater capture yield than the DNA corresponding to the second target region set.

A “target region set” or “set of target regions” refers to a plurality of genomic loci targeted for capture and/or targeted by a set of probes (e.g., through sequence complementarity).

“Corresponding to a target region set” means that a nucleic acid, such as cfDNA, originated from a locus in the target region set or specifically binds one or more probes for the target region set.

“Specifically binds” in the context of a probe or other oligonucleotide and a target sequence means that under appropriate hybridization conditions, the oligonucleotide or probe hybridizes to its target sequence, or replicates thereof, to form a stable probe: target hybrid, while at the same time formation of stable probe:non-target hybrids is minimized. Thus, a probe hybridizes to a target sequence or replicate thereof to a sufficiently greater extent than to a non-target sequence, to enable capture or detection of the target sequence. Appropriate hybridization conditions are well-known in the art, may be predicted based on sequence composition, or can be determined by using routine testing methods (see, e.g., Sambrook et al., Molecular Cloning, A Laboratory Manual, 2nd ed. (Cold Spring Harbor Laboratory Press, Cold Spring Harbor, NY, 1989) at §§ 1.90-1.91, 7.37-7.57, 9.47-9.51 and 11.47-11.57, particularly §§ 9.50-9.51, 11.12-11.13, 11.45-11.47 and 11.55-11.57, incorporated by reference herein). “Specifically binds” in the context of a protein and its binding partner means that under appropriate conditions, the protein binds to its binding partner to form a stable binding interaction, while at the same time formation of stable binding interactions with other molecules is minimized. Thus, a protein (e.g., an antibody) that specifically binds to its binding partner (e.g., a target protein) binds to the binding partner to a sufficiently greater extent than to other, non-binding partner proteins to enable capture or detection of the binding partner protein.

“Sequence-variable target region set” refers to a set of target regions that may exhibit changes in sequence such as nucleotide substitutions (i.e., single nucleotide variations), insertions, deletions, or gene fusions or transpositions in neoplastic cells (e.g., tumor cells and cancer cells) relative to normal cells.

“Epigenetic target region set” refers to a set of target regions that may show sequence-independent changes in neoplastic cells (e.g., tumor cells and cancer cells) relative to normal cells or that may show sequence-independent changes in cfDNA from subjects having cancer relative to cfDNA from healthy subjects. Examples of sequence-independent changes include, but are not limited to, changes in methylation (increases or decreases), nucleosome distribution, cfDNA fragmentation patterns, CCCTC-binding factor (“CTCF”) binding, transcription start sites, and regulatory protein binding regions. Epigenetic target region sets thus include, but are not limited to, hypermethylation variable target region sets, hypomethylation variable target region sets, and fragmentation variable target region sets, such as CTCF binding sites and transcription start sites. For present purposes, loci susceptible to neoplasm-, tumor-, or cancer-associated focal amplifications and/or gene fusions may also be included in an epigenetic target region set because detection of a change in copy number by sequencing or a fused sequence that maps to more than one locus in a reference genome tends to be more similar to detection of exemplary epigenetic changes discussed herein than detection of nucleotide substitutions, insertions, or deletions, e.g., in that the focal amplifications and/or gene fusions can be detected at a relatively shallow depth of sequencing because their detection does not depend on the accuracy of base calls at one or a few individual positions.

A nucleic acid is “produced by a tumor” or is “circulating tumor DNA” (“ctDNA”) if it originated from a tumor cell. Tumor cells are neoplastic cells that originated from a tumor, regardless of whether they remain in the tumor or become separated from the tumor (as in the cases, e.g., of metastatic cancer cells and circulating tumor cells).

The term “methylation” or “DNA methylation” refers to addition of a methyl group to a nucleobase in a nucleic acid molecule. In some embodiments, methylation refers to addition of a methyl group to a cytosine at a CpG site (cytosine-phosphate-guanine site (i.e., a cytosine followed by a guanine in a 5′→3′ direction of the nucleic acid sequence). In some embodiments, DNA methylation refers to addition of a methyl group to adenine, such as in N⁶-methyladenine. In some embodiments, DNA methylation is 5-methylation (modification of the 5th carbon of the 6-carbon ring of cytosine). In some embodiments, 5-methylation refers to addition of a methyl group to the 5C position of the cytosine to create 5-methylcytosine (5mC). In some embodiments, methylation comprises a derivative of 5mC. Derivatives of 5mC include, but are not limited to, 5-hydroxymethylcytosine (5hmC), 5-formylcytosine (5fC), and 5-caryboxylcytosine (5caC). In some embodiments, DNA methylation is 3C methylation (modification of the 3rd carbon of the 6-carbon ring of cytosine). In some embodiments, 3C methylation comprises addition of a methyl group to the 3C position of the cytosine to generate 3-methylcytosine (3mC). Methylation can also occur at non CpG sites, for example, methylation can occur at a CpA, CpT, or CpC site. DNA methylation can change the activity of methylated DNA region. For example, when DNA in a promoter region is methylated, transcription of the gene may be repressed. DNA methylation is critical for normal development and abnormality in methylation may disrupt epigenetic regulation. The disruption, e.g., repression, in epigenetic regulation may cause diseases, such as cancer. Promoter methylation in DNA may be indicative of cancer

The term “hypermethylation” refers to an increased level or degree of methylation of nucleic acid molecule(s) relative to the other nucleic acid molecules within a population (e.g., sample) of nucleic acid molecules. In some embodiments, hypermethylated DNA can include DNA molecules comprising at least 1 methylated residue, at least 2 methylated residues, at least 3 methylated residues, at least 5 methylated residues, or at least 10 methylated residues.

The term “hypomethylation” refers to a decreased level or degree of methylation of nucleic acid molecule(s) relative to the other nucleic acid molecules within a population (e.g., sample) of nucleic acid molecules. In some embodiments, hypomethylated DNA includes unmethylated DNA molecules. In some embodiments, hypomethylated DNA can include DNA molecules comprising 0 methylated residues, at most 1 methylated residue, at most 2 methylated residues, at most 3 methylated residues, at most 4 methylated residues, or at most 5 methylated residues.

The term “agent that recognizes a cell debris marker, a chromatin-associated target, or a nucleic acid modification” refers to a molecule or reagent that specifically binds to or specifically detects one or more cell debris markers, chromatin-associated targets, or nucleic acid modifications. In some embodiments, a nucleic acid modification comprises a modified nucleobase. A “modified nucleobase” is a nucleobase that comprises a difference in chemical structure from an unmodified nucleobase. In the case of DNA, an unmodified nucleobase is adenine, cytosine, guanine, or thymine. In some embodiments, a modified nucleobase is a modified cytosine. In some embodiments, a modified nucleobase is a methylated nucleobase. In some embodiments, a modified cytosine is a methyl cytosine, e.g., a 5-methylcytosine. In such embodiments, the cytosine modification is a methyl. Agents that recognize a methyl cytosine in DNA include but are not limited to “methyl binding reagents,” which refer herein to reagents that bind to a methyl cytosine. Methyl binding reagents include but are not limited to methyl binding domains (MBDs) and methyl binding proteins (MBPs) and antibodies specific for methyl cytosine. In some embodiments, such antibodies bind to 5-methyl cytosine in DNA. In some such embodiments, the DNA may be single-stranded or double-stranded.

The terms “disease” and “disease state” encompass disorders and conditions not present in a healthy subject. Diseases include infections and conditions associated with undesired losses or gains of function (e.g., organ failure; autoimmune conditions; cancer).

As used herein, “organ failure” means the undesired loss of function of an organ. In some embodiments, the level of a tissue-specific cell material can be indicative of organ failure, e.g., the level of cell debris, apoptotic bodies, or other cell material consistent with cell death can correlate with loss of organ function.

As used herein, “tissue-specific” in the context of a biomolecule or cell material refers to a property of the biomolecule or cell material that is specific to one or more cell or tissue types. Such properties may be independent of whether the biomolecule or cell material is from a cell or tissue in a healthy condition or in a diseased condition. Tissue-specific properties may include but are not limited to sequences and differential modifications, such as differentially methylated regions of nucleic acids and differentially post-translationally modified proteins.

As used herein, “tissue-specific hydroxymethylated DNA” means a DNA molecule comprising one or more hydroxymethyl modifications, wherein the DNA sequence and/or hydroxymethylation modification pattern are specific to one or more cell or tissue types.

As used herein, “tissue-specific fragmented DNA” means a fragmented DNA molecule, wherein the DNA sequence and/or fragmentation pattern are specific to one or more cell or tissue types.

As used herein, “tissue-specific modified histone” means a histone comprising one or more modifications relative to a histone consisting of unmodified linked amino acids, such as a post-translational modification, wherein the sequence with which the histone is associated in combination with the identity of the modification and/or location of the modification on the histone are specific to one or more cell or tissue types.

As used herein, “tissue-specific bacterial nucleic acid” means a nucleic acid molecule from a bacterial cell comprising a sequence that is specific to bacterial cells that are specifically located in one or more tissue types or organs in a human or other mammalian body.

As used herein, “tissue-specific protein or cell debris” means proteins or other components of dead cells that can be released into the blood or other bodily fluids following apoptosis, autophagic cell death, necrosis, or other types of cell death and that have a property (e.g., amino acid sequence and/or post-translational modification or combination of sequences and/or modifications) specific to one or more cell or tissue types.

As used herein, “tissue-specific extracellular vesicle” means a membrane bound, enclosed body, e.g., an apoptotic body or exosome, that can be released from a cell, e.g., a living, apoptotic, or necrotic cell, having a property (e.g., a component or combination of components or an amino acid sequence of a component protein and/or post-translational modification thereof or combination of sequences and/or modifications) that is specific to one or more cell or tissue types.

As used herein, “tissue-specific RNA” means a RNA molecule having a property (e.g., sequence or post-transcriptional modification) that is specific to one or more cell or tissue types.

The terms “or a combination thereof” and “or combinations thereof” as used herein refers to any and all permutations and combinations of the listed terms preceding the term. For example, “A, B, C, or combinations thereof” is intended to include at least one of: A, B, C, AB, AC, BC, or ABC, and if order is important in a particular context, also BA, CA, CB, ACB, CBA, BCA, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

“Or” is used in the inclusive sense, i.e., equivalent to “and/or,” unless the context requires otherwise.

### II. Exemplary Methods

### A. Methods of Detecting Metastasis

Methods of detecting metastasis disclosed herein include methods comprising detecting tissue-specific cell material released from a potential metastasis site in a sample from a subject. Without wishing to be bound by theory, when a metastasis invades a distal, healthy tissue, the invaded tissue of the metastasis site may experience increased apoptosis and/or shedding as a result of the invasion. Thus, cell materials of the metastasis site may be released at higher levels than they are released in the absence of the metastasis. Although such cell materials may not comprise aberrant features or features related to disease (i.e., the cell materials may have so-called “normal” features), some such cell materials may comprise tissue-specific features that allow identification of the tissue of origin of the materials. If the levels of such “normal” tissue-specific cell materials is greater than expected in the absence of invasion by metastasis, is higher than expected relative to a certain comparator cell material, or increases over time, a metastasis may be present. Even higher than expected levels of tissue-specific materials may be difficult to detect. In some embodiments, the methods herein comprise detection of tissue-specific cell materials and quantification of the levels of such tissue-specific cell materials with enough sensitivity and precision in order to detect metastasis and to identify the tissue type of the metastasis site.

In some embodiments, the methods herein comprise detecting the presence or absence of metastasis based at least in part on the detected presence or level of at least one cell material released from a potential metastasis site. In such embodiments, the methods may comprise additional steps, e.g., steps to confirm the presence of the metastasis. For example, such methods may comprise imaging the potential metastasis site.

In some embodiments, the methods herein comprise detecting the presence or level of at least one cell material released from a potential metastasis site. In some embodiments, the at least one cell material is not present or is present at levels below the limit of detection of the methods herein in the absence of metastasis. In some such embodiments, the presence but not the level of at least one cell material is detected. In some embodiments, detecting the level of at least one cell material is necessary to facilitate detection of metastasis. In some embodiments, the absolute level of at least one cell material is detected. In some embodiments, the absolute level of at least one cell material is detected, and the absolute level is compared to a reference level of the same cell material, to the absolute level of the same cell material detected in the same subject at a different time, or to the level of a different cell material. Some such embodiments comprise detecting whether the presence or level of at least one cell material released from a potential metastasis site has changed in comparison to or relative to the level detected in a previous sample. In some embodiments, the previous sample was obtained 1-60 months before the sample was obtained. In some embodiments, the previous sample was obtained 3, 6, 9, 12, 18, 24, 36, 48, or 60 months before the sample was obtained. In some embodiments, the previous sample was obtained near the time of the diagnosis of the primary cancer, tumor, or neoplasm. In some embodiments, the previous sample was obtained within one month of the diagnosis of the primary cancer, tumor, or neoplasm. In such embodiments, “within one month” means the time period from one month before the diagnosis up to one month after the diagnosis. In some embodiments, the primary cancer, tumor, or neoplasm was not metastatic at the time the previous sample was obtained. In some embodiments, the previous sample was obtained before the subject underwent treatment for the primary cancer, tumor, or neoplasm. In some embodiments, the sample was obtained after the subject underwent treatment for the primary cancer, tumor, or neoplasm. In some embodiments, the previous sample was obtained when the subject did not have a cancer, tumor, or neoplasm. In some such embodiments, the previous sample was obtained as part of a cancer screening. In some embodiments, the sample was obtained as part of a cancer screening. In some embodiments, the primary cancer, tumor, or neoplasm and the presence of a metastasis are detected in the sample.

In some embodiments, a relative level of at least one cell material is detected. In some such embodiments, the relative level is relative to the level of a comparator cell material. In some embodiments, the comparator cell material is an autologous comparator cell material. In some such embodiments, the comparator cell material is present in the same sample as the cell material released from the potential metastasis site. In some embodiments, the comparator cell material is present in a sample obtained from the subject before being diagnosed with a cancer, tumor, or neoplasm or when the subject did not have a metastasis. In some embodiments, the comparator cell material is a heterologous comparator cell material. In some such embodiments, the level of the comparator cell material is determined based on an average of levels of the comparator cell material detected in samples obtained from a reference population. In some such embodiments, the reference population is a population of healthy subjects, such as subjects that do not have a cancer, tumor, or neoplasm. Some embodiments herein comprise detecting the relative level of at least one cell material, wherein the relative level is the level relative to the level of a comparator cell material, and detecting whether the relative level has changed in comparison to the relative level detected in a previous sample. In some embodiments, the comparator cell material is a material released from a cell or tissue type that is not a candidate for a potential metastasis site. In some embodiments, the comparator cell material comprises cell material released from erythroid cells or tissue, or a healthy cell or tissue type. In some embodiments, the comparator cell material comprises cell material released from the primary cancer, tumor, or neoplasm. In some embodiments, the comparator cell material comprises the same type or types of cell materials detected from the potential metastasis site except that the comparator cell material is specific for a different tissue than that of the potential metastasis site. For example, the cell material from the potential metastasis site may be colon-specific methylated cfDNA, and the comparator cell material may be erythroid-specific methylated cfDNA.

Methods herein comprise detecting cell material released from a potential metastasis site. In some embodiments, the cell material released from the potential metastasis site does not comprise methylated cfDNA. In some embodiments, the cell material released from the potential metastasis site comprises methylated cfDNA. In some embodiments, the cell material released from the potential metastasis site comprises a plurality of cell materials. In some embodiments, the plurality of cell materials released from the potential metastasis site comprises methylated cfDNA and one or more other types of tissue-specific cell material. In some embodiments, the cell material released from the potential metastasis site comprises cell-free cell material. In some embodiments, the cell material released from the potential metastasis site comprises hydroxymethylated DNA, fragmented DNA, modified histones, or other chromatin-associated targets, bacterial nucleic acids, proteins, such as post-translationally modified proteins, cell debris, lipids, such as membrane-specific lipids, or RNA. In some embodiments, the DNA is cfDNA. In some embodiments, the cell material released from the potential metastasis site comprises a tissue-specific epigenetic target region set. In some embodiments, the cell material released from the potential metastasis site consists of tissue-specific cell materials. In some embodiments, the cell material released from the potential metastasis site is specific to the brain, lung, skin, nose, throat, liver, bone, bone marrow, pancreas, lymph nodes, bowel, rectum, colon, prostate, thyroid, bladder, head, neck, kidney, mouth, stomach, or spleen.

In some embodiments, the cell material released from the potential metastasis site comprises bacterial nucleic acids. In some such embodiments, the bacterial nucleic acids are cell-free bacterial nucleic acids. In some embodiments, the bacterial nucleic acids are specific to one or more bacterial species. In some embodiments, the bacterial nucleic acids are tissue-specific, such as bacterial nucleic acids that are specific to one or more bacterial species that are located in specific tissue types. For example, in some embodiments, the bacterial nucleic acids are specific to bacterial species located in the colon and are therefore colon-specific bacterial nucleic acids. In some embodiments, the bacterial nucleic acids are specific to species associated with cancer. In some embodiments, the bacterial nucleic acids are specific to symbiotic or harmless bacterial species. In some embodiments, the bacterial nucleic acids comprise 16S rRNA or genes encoding 16S rRNA. In some embodiments, the bacterial nucleic acids comprise a nucleic acid encoding a gene specific to Enterotoxigenic Bacteroides fragilis, Fusobacterium nucleatum, Bilophila wadsworthia, Streptococcus bovis, Helicobacter pylori, Bacteroides fragilis, or Clostridium septicum.

In some embodiments, the cell material released from the potential metastasis site comprises chromatin-associated targets, such as tissue-specific modified histones. In some embodiments, the chromatin-associated targets comprise histone variants, post-translationally modified histones, and proteins other than histones that are part of or bind to chromatin. In some embodiments, the chromatin-associated targets comprise a post-translational histone modification. In some embodiments, the post-translational histone modification is acetylation (Ac), methylation (me), dimethylation (me2), trimethylation (me3), phosphorylation, ubiquitylation, ADP-ribosylation, crotonylation, succinylation, or malonylation. Such modifications may be described using an abbreviation identifying the histone comprising the modification, the amino acid of the histone comprising the modification, and the identity of the modification. For example, H3K4me2 indicates that the post-translational modification is dimethylation (“me2”) of lysine 4 (“K4”) of core histone 3 (“H3”). In some embodiments, the chromatin-associated targets comprise one or more of post-translational histone modifications H3K4me2, H3K4me3, H3K9Ac, H3K9me3, H3K27Ac, HeK27me3, and H3K36me3. In some embodiments, the chromatin-associated targets comprise a histone variant, such as H3.1, H3.3, or H2A.Z. In some embodiments, the chromatin-associated target is a post-translational modification of a histone variant. In some embodiments, the chromatin-associated targets comprise at least one protein other than a histone, including but not limited to RNA polymerase II, CTCF, Yin Yang 1 (YY1), and nuclear receptors. In some such embodiments, the nuclear receptor is estrogen receptor (ER), androgen receptor (AR), a peroxisome proliferator-activated receptor (PPAR), liver X receptor alpha (LXR), retinoic acid receptor alpha (RAR), farnesoid X receptor (FXR), pregnane X receptor (PXR), thyroid hormone receptor (THR), vitamin D receptor (VDR), or retinoid X receptor (RXR).

In some embodiments, the cell material released from the potential metastasis site comprises cell debris. In some embodiments, the cell debris comprises a tissue-specific cell material, such as a tissue-specific protein or tissue-specific post-translationally modified protein. In some embodiments, the cell debris comprises PD-L1, CTLA4, NYESO1, mesothelin, CA15-3, CA19-9, CA-125, CA-172-4, or a cell material released from an exosome. In some embodiments, the cell debris is detected by contacting the sample with a molecule or agent that binds a cell debris marker that is associated with the cell debris before identifying the cell debris by another method, such as mass spectrometry, ELISA, multiplex immunoassay, western blot, an electrochemiluminescent (ECL) assay, flow cytometry, PCR, or sequencing. In some embodiments, the agent that binds a cell debris marker is Annexin V or an antibody that specifically binds to phosphatidylserine. In some embodiments, the agent that binds a cell debris marker is an antibody, aptamer, nanobody, affimer, or DARpin.

In some embodiments, the cell material released from the potential metastasis site comprises a tissue-specific RNA. In some embodiments, the RNA is a microRNA, exosomal RNA, or extracellular RNA.

In some embodiments, the potential metastasis site is any tissue type that is not the same tissue type of the primary cancer, tumor, or neoplasm. In some embodiments, the potential metastasis site is a likely or known potential site of metastasis for patients having the type or primary cancer, tumor, or neoplasm with which the subject has been diagnosed. Alternatively, in some embodiments, the type of the primary cancer is unknown. In some embodiments, the potential metastasis site is brain, lung, skin, nose, throat, liver, bone, bone marrow, pancreas, lymph nodes, bowel, rectum, colon, prostate, thyroid, bladder, head, neck, kidney, mouth, stomach, or spleen. In some embodiments, the presence of a metastasis is detected at the potential metastasis site, and the potential metastasis site is an actual metastasis site. In some embodiments, identification of the tissue type from which the cell material was released facilitates identification of the tissue type and/or location of the metastasis site. For example, identification of lung-specific cell material may indicate that a primary cancer has metastasized to the lung.

In some embodiments, the presence of absence of metastasis is detected at or near the same time that a primary cancer, tumor, or neoplasm is detected. For example, a primary cancer, tumor, or neoplasm and the presence or absence of metastasis may be detected from the same sample. In some such embodiments, the primary cancer, tumor, or neoplasm is detected using methods of detecting cell materials associated with cancer described herein or elsewhere. In some embodiments, the primary cancer, tumor, or neoplasm is a cancer, tumor, or neoplasm that is known to be potentially metastatic. In some embodiments, the primary cancer, tumor, or neoplasm is a lymphoma, a leukemia, multiple myeloma, or a cancer, tumor, or neoplasm of the liver, skin, lung, breast, or pancreas.

Some embodiments comprise detecting metastasis-associated sequence variants of nucleic acids released from a primary cancer, tumor, or neoplasm. In some such embodiments, the nucleic acids comprising the metastasis-associated sequence variants are tissue specific, thus facilitating identification of the tissue type of the primary cancer, tumor, or neoplasm and detection of the presence of a metastasis. Exemplary metastasis-associated sequence variants are described, e.g., in Aljohani et al., Mutagenesis 33:137-145 (2018).

Methods disclosed herein comprise detecting cell materials released from potential metastasis sites. In some embodiments, the detecting comprises amplifying nucleic acids released from the potential metastasis site by quantitative PCR (qPCR), reverse transcription qPCR, (RT-PCR), or digital PCR, such as ddPCR. In some embodiments, the detecting comprises sequencing DNA released from the potential metastasis site. In some embodiments, the detecting comprises immunoprecipitation, mass spectrometry, ELISA, multiplex immunoassay, western blot, electrochemiluminescent (ECL) assay, flow cytometry, or chromatography. In some embodiments, the detecting comprises steps described elsewhere herein.

### B. Subjects

In some embodiments, the sample is obtained from a subject having a cancer, precancer, tumor, or neoplasm. In some embodiments, the sample is obtained from a subject having or suspected of having a metastasis of the cancer, tumor, or neoplasm. In some embodiments, the sample is obtained from a subject suspected of having a cancer or a precancer. In some embodiments, the sample is obtained from a healthy subject or a subject not known to have a cancer, precancer, tumor, or neoplasm. In some embodiments, the sample is obtained from a subject in remission from a tumor, cancer, or neoplasm (e.g., following chemotherapy, surgical resection, radiation, or a combination thereof). In some embodiments, the precancer, cancer, tumor, or neoplasm or suspected precancer, cancer, tumor, or neoplasm is of the bladder, head and neck, lung, colon, rectum, kidney, breast, prostate, skin, or liver. In some embodiments, the precancer, cancer, tumor, or neoplasm or suspected precancer, cancer, tumor, or neoplasm is of the lung. In some embodiments, the precancer, cancer, tumor, or neoplasm or suspected precancer, cancer, tumor, or neoplasm is of the colon or rectum. In some embodiments, the precancer, cancer, tumor, or neoplasm or suspected precancer, cancer, tumor, or neoplasm is of the breast. In some embodiments, the precancer, cancer, tumor, or neoplasm or suspected precancer, cancer, tumor, or neoplasm is of the prostate. In any of the foregoing embodiments, the subject may be a human subject. In some embodiments, the sample is obtained from a subject having a stage I cancer, stage II cancer, stage III cancer or stage IV cancer.

In some embodiments, the subject is a human, a mammal, an animal, a companion animal, a service animal, or a pet. The subject may have a cancer, precancer, infection, transplant rejection, or other disease or disorder related to changes in the immune system. The subject may not have cancer or a detectable cancer symptom or a detectable symptom of metastasis. The subject may have been treated with one or more cancer therapy, e.g., any one or more of chemotherapies, antibodies, vaccines or biologic therapeutics. The subject may be in remission. The subject may or may not be diagnosed of being susceptible to cancer or any cancer-associated genetic mutations/disorders.

### C. Samples

A sample can be any biological sample isolated from a subject. A sample can be a bodily sample. Samples can include body tissues or fluids, such as known or suspected solid tumors, whole blood, platelets, serum, plasma, stool, red blood cells, white blood cells or leucocytes, endothelial cells, tissue biopsies, cerebrospinal fluid synovial fluid, lymphatic fluid, ascites fluid, interstitial or extracellular fluid, the fluid in spaces between cells, gingival crevicular fluid, bone marrow, pleural effusions, pleura fluid, cerebrospinal fluid, saliva, mucous, sputum, semen, sweat, and urine. Samples are preferably body fluids, particularly blood and fractions thereof, cerebrospinal fluid, pleura fluid, saliva, sputum, or urine. A sample can be in the form originally isolated from a subject or can have been subjected to further processing to remove or add components, such as cells, or enrich for one component relative to another. Thus, a preferred body fluid for analysis is plasma or serum comprising cell-free nucleic acids.

In some embodiments, cell materials are obtained from a serum, plasma or blood sample from a subject suspected of having neoplasm, a tumor, precancer, or cancer or a metastasis of a neoplasm, a tumor, or cancer. A sample can be isolated or obtained from a subject and transported to a site of sample analysis. The sample may be preserved and shipped at a desirable temperature, e.g., room temperature, 4° C., −20° C., and/or −80° C. A sample can be isolated or obtained from a subject at the site of the sample analysis.

In some embodiments, the sample comprises plasma. The volume of plasma obtained can depend on the desired read depth for sequenced regions. Exemplary volumes are 0.4-40 ml, 5-20 ml, 10-20 ml. For examples, the volume can be 0.5 mL, 1 mL, 5 mL 10 mL, 20 mL, 30 mL, or 40 mL. A volume of sampled plasma may be 5 to 20 mL.

### D. Adapter Ligation or Addition; Tagging

In some embodiments, the cell material released from the potential metastasis site, a comparator cell material, or other cell material in the sample comprises DNA. In some such embodiments, the disclosed methods comprise adding adapters to the DNA. In some embodiments, adapters may be added to DNA concurrently with an amplification procedure, e.g., by providing the adapters in a 5′ portion of a primer (where PCR is used, this can be referred to as library prep-PCR or LP-PCR), before, of after an amplification step. In some embodiments, adapters are added by other approaches, such as ligation. In some such methods, first adapters are added to the nucleic acids by ligation to the 3′ ends thereof, which may include ligation to single-stranded DNA. The adapter can be used as a priming site for second-strand synthesis, e.g., using a universal primer and a DNA polymerase. A second adapter can then be ligated to at least the 3′ end of the second strand of the now double-stranded molecule. In some embodiments, the first adapter comprises an affinity tag, such as biotin, and nucleic acid ligated to the first adapter is bound to a solid support (e.g., bead), which may comprise a binding partner for the affinity tag such as streptavidin. For further discussion of a related procedure, see Gansauge et al., Nature Protocols 8:737-748 (2013). Commercial kits for sequencing library preparation compatible with single-stranded nucleic acids are available, e.g., the Accel-NGS® Methyl-Seq DNA Library Kit from Swift Biosciences. In some embodiments, after adapter ligation, nucleic acids are amplified. In some embodiments, end repair of the DNA is performed prior to addition of adapters.

Preferably, the adapters include different tags of sufficient numbers that the number of combinations of tags results in a low probability e.g., 95, 99 or 99.9% of two nucleic acids with the same start and stop points receiving the same combination of tags. Adapters, whether bearing the same or different tags, can include the same or different primer binding sites, but preferably adapters include the same primer binding site.

In some embodiments, following attachment of adapters, the nucleic acids are subject to amplification. The amplification can use, e.g., universal primers that recognize primer binding sites in the adapters.

In some embodiments, following attachment of adapters, the DNA or a subsample comprising a portion of the DNA is partitioned, as described elsewhere herein. The DNA may be partitioned into at least two partitioned subsamples differing in the extent to which the DNA bears a certain modification or feature. Alternatively, partitioning may be performed before adapter attachment, in which case the adapters may comprise differential tags that include a component that identifies in which partition a molecule was present.

In some embodiments, DNA is linked at both ends to Y-shaped adapters including primer binding sites and tags. In some such embodiments, the DNA is amplified.

Tagging DNA molecules is a procedure in which a tag is attached to or associated with the DNA molecules. Such tags can be molecules, such as nucleic acids, containing information that indicates a feature of the molecule with which the tag is associated. Tags can allow one to differentiate molecules from which sequence reads originated. For example, molecules can bear a sample tag (which distinguishes molecules in one sample from those in a different sample) or a molecular tag/molecular barcode/barcode (which distinguishes different molecules from one another (in both unique and non-unique tagging scenarios). For methods that involve a partitioning step, a partition tag (which distinguishes molecules in one partition from those in a different partition) may be included. In some embodiments, adapters added to DNA molecules comprise tags. In certain embodiments, a tag can comprise one or a combination of barcodes. As used herein, the term “barcode” refers to a nucleic acid molecule having a particular nucleotide sequence, or to the nucleotide sequence, itself, depending on context. A barcode can have, for example, between 10 and 100 nucleotides. A collection of barcodes can have degenerate sequences or can have sequences having a certain hamming distance, as desired for the specific purpose. So, for example, a molecular barcode can be comprised of one barcode or a combination of two barcodes, each attached to different ends of a molecule. Additionally or alternatively, for different partitions and/or samples, different sets of molecular barcodes, or molecular tags can be used such that the barcodes serve as a molecular tag through their individual sequences and also serve to identify the partition and/or sample to which they correspond based the set of which they are a member. Tags comprising barcodes can be incorporated into or otherwise joined to adapters. Tags can be incorporated by ligation, overlap extension PCR among other methods.

Tagging strategies can be divided into unique tagging and non-unique tagging strategies. In unique tagging, all or substantially all of the molecules in a sample bear a different tag, so that reads can be assigned to original molecules based on tag information alone. Tags used in such methods are sometimes referred to as “unique tags”. In non-unique tagging, different molecules in the same sample can bear the same tag, so that other information in addition to tag information is used to assign a sequence read to an original molecule. Such information may include start and stop coordinate, coordinate to which the molecule maps, start or stop coordinate alone, etc. Tags used in such methods are sometimes referred to as “non-unique tags”. Accordingly, it is not necessary to uniquely tag every molecule in a sample. It suffices to uniquely tag molecules falling within an identifiable class within a sample. Thus, molecules in different identifiable families can bear the same tag without loss of information about the identity of the tagged molecule.

In certain embodiments of non-unique tagging, the number of different tags used can be sufficient that there is a very high likelihood (e.g., at least 99%, at least 99.9%, at least 99.99% or at least 99.999% that all molecules of a particular group bear a different tag. It is to be noted that when barcodes are used as tags, and when barcodes are attached, e.g., randomly, to both ends of a molecule, the combination of barcodes, together, can constitute a tag. This number, in term, is a function of the number of molecules falling into the calls. For example, the class may be all molecules mapping to the same start-stop position on a reference genome. The class may be all molecules mapping across a particular genetic locus, e.g., a particular base or a particular region (e.g., up to 100 bases or a gene or an exon of a gene). In certain embodiments, the number of different tags used to uniquely identify a number of molecules, z, in a class can be between any of 2*z, 3*z, 4*z, 5*z, 6*z, 7*z, 8*z, 9*z, 10*z, 11 *z, 12*z, 13*z, 14*z, 15*z, 16*z, 17*z, 18*z, 19*z, 20*z or 100*z (e.g., lower limit) and any of 100,000*z, 10,000*z, 1000*z or 100*z (e.g., upper limit).

For example, in a sample of about 5 ng to 30 ng of cell free DNA, one expects around 3000 molecules to map to a particular nucleotide coordinate, and between about 3 and 10 molecules having any start coordinate to share the same stop coordinate. Accordingly, about 50 to about 50,000 different tags (e.g., between about 6 and 220 barcode combinations) can suffice to uniquely tag all such molecules. To uniquely tag all 3000 molecules mapping across a nucleotide coordinate, about 1 million to about 20 million different tags would be required.

Generally, assignment of unique or non-unique tags barcodes in reactions follows methods and systems described by US patent applications 20010053519, 20030152490, 20110160078, and U.S. Pat. Nos. 6,582,908 and 7,537,898 and 9,598,731. Tags can be linked to sample nucleic acids randomly or non-randomly.

In some embodiments, the tagged nucleic acids are sequenced after loading into a microwell plate. The microwell plate can have 96, 384, or 1536 microwells. In some cases, they are introduced at an expected ratio of unique tags to microwells. For example, the unique tags may be loaded so that more than about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 unique tags are loaded per genome sample. In some cases, the unique tags may be loaded so that less than about 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 unique tags are loaded per genome sample. In some cases, the average number of unique tags loaded per sample genome is less than, or greater than, about 1, 2, 3, 4, 5, 6, 7, 8, 9, 10, 20, 50, 100, 500, 1000, 5000, 10000, 50,000, 100,000, 500,000, 1,000,000, 10,000,000, 50,000,000 or 1,000,000,000 unique tags per genome sample.

A preferred format uses 20-50 different tags (e.g., barcodes) ligated to both ends of target nucleic acids. For example 35 different tags (e.g., barcodes) ligated to both ends of target molecules creating 35×35 permutations, which equals 1225 for 35 tags. Such numbers of tags are sufficient so that different molecules having the same start and stop points have a high probability (e.g., at least 94%, 99.5%, 99.99%, 99.999%) of receiving different combinations of tags. Other barcode combinations include any number between 10 and 500, e.g., about 15×15, about 35×35, about 75×75, about 100×100, about 250×250, about 500×500.

In some cases, unique tags may be predetermined or random or semi-random sequence oligonucleotides. In other cases, a plurality of barcodes may be used such that barcodes are not necessarily unique to one another in the plurality. In this example, barcodes may be ligated to individual molecules such that the combination of the barcode and the sequence it may be ligated to creates a unique sequence that may be individually tracked. As described herein, detection of non-unique barcodes in combination with sequence data of beginning (start) and end (stop) portions of sequence reads may allow assignment of a unique identity to a particular molecule. The length or number of base pairs, of an individual sequence read may also be used to assign a unique identity to such a molecule. As described herein, fragments from a single strand of nucleic acid having been assigned a unique identity, may thereby permit subsequent identification of fragments from the parent strand.

In some embodiments, two or more populations, samples, subsamples, or partitions are differentially tagged. Tags can be used to label distinct DNA populations in order to correlate the tag (or tags) with a specific population or partition. In some embodiments, a single tag can be used to label a specific population or partition. In some embodiments, multiple different tags can be used to label a specific population or partition. In embodiments employing multiple different tags to label a specific partition, the set of tags used to label one partition can be readily differentiated for the set of tags used to label other partitions. In some embodiments, the tags may have additional functions, for example the tags can be used to index sample sources or used as unique molecular identifiers (which can be used to improve the quality of sequencing data by differentiating sequencing errors from mutations, for example as in Kinde et al., Proc Nat'l Acad Sci USA 108:9530-9535 (2011), Kou et al., PLOS ONE, 11: e0146638 (2016)) or used as non-unique molecule identifiers, for example as described in U.S. Pat. No. 9,598,731. Similarly, in some embodiments, the tags may have additional functions, for example the tags can be used to index sample sources or used as non-unique molecular identifiers (which can be used to improve the quality of sequencing data by differentiating sequencing errors from mutations).

In some embodiments, partition tagging comprises tagging molecules in each partition with a partition tag. After re-combining partitions (e.g., to reduce the number of sequencing runs needed and avoid unnecessary cost) and sequencing molecules, the partition tags identify the source partition. In another embodiment, different partitions are tagged with different sets of molecular tags, e.g., comprised of a pair of barcodes. In this way, each molecular barcode indicates the source partition as well as being useful to distinguish molecules within a partition. For example, a first set of 35 barcodes can be used to tag molecules in a first partition, while a second set of 35 barcodes can be used tag molecules in a second partition.

In some embodiments, after tagging, the molecules may be pooled for sequencing in a single run. In some embodiments, a sample tag is added to the molecules, e.g., in a step subsequent to addition of other tags and pooling. Sample tags can facilitate pooling material generated from multiple samples for sequencing in a single sequencing run.

In some embodiments, partition tags may be correlated to the sample as well as the partition. As a simple example, a first tag can indicate a first partition of a first sample; a second tag can indicate a second partition of the first sample; a third tag can indicate a first partition of a second sample; and a fourth tag can indicate a second partition of the second sample.

While tags may be attached to molecules based on one or more characteristics, the final tagged molecules in the library may no longer possess that characteristic. For example, while single-stranded DNA molecules may be partitioned and/or tagged, the final tagged molecules in the library are likely to be double stranded. Similarly, while DNA may be subject to partition based on different levels of methylation, in the final library, tagged molecules derived from these molecules are likely to be unmethylated. Accordingly, the tag attached to molecule in the library typically indicates the characteristic of the “parent molecule” from which the ultimate tagged molecule is derived, not necessarily to characteristic of the tagged molecule, itself.

As an example, barcodes 1, 2, 3, 4, etc. are used to tag and label molecules in the first partition; barcodes A, B, C, D, etc. are used to tag and label molecules in the second partition; and barcodes a, b, c, d, etc. are used to tag and label molecules in the third partition. Differentially tagged partitions can be pooled prior to sequencing. Differentially tagged partitions can be separately sequenced or sequenced together concurrently, e.g., in the same flow cell of an Illumina sequencer.

After sequencing, analysis of reads can be performed on a partition-by-partition level, as well as a pooled DNA level. Tags are used to sort reads from different partitions. Analysis can include in silico analysis to determine genetic and epigenetic variation (e.g., methylation, chromatin structure, etc.) using sequence information, genomic coordinates length, coverage, and/or copy number.

### E. Amplification

In some embodiments, the sample comprises nucleic acids that are amplified. For example, DNA flanked by adapters added to the DNA as described herein can be amplified by PCR or other amplification methods. In some embodiments, amplification is primed by primers binding to primer binding sites in adapters flanking a DNA molecule to be amplified. Amplification methods can involve cycles of denaturation, annealing and extension, resulting from thermocycling or can be isothermal as in transcription-mediated amplification. Other amplification methods include the ligase chain reaction, strand displacement amplification, nucleic acid sequence based amplification, and self-sustained sequence based replication.

In some embodiments, detecting nucleic acids comprises amplification, such as embodiments comprising reverse transcription of RNA, qPCR or digital PCR. Some such embodiments comprising sequencing nucleic acids using qPCR or digital PCR do not comprise standard DNA library preparation steps, such as adapter ligation or tagging.

In some embodiments, dsDNA ligations with T-tailed and C-tailed adapters can be performed, which result in amplification of at least 50, 60, 70 or 80% of double stranded nucleic acids before linking to adapters.

### F. Sequencing

In some embodiments, the detection of nucleic acid sequences comprises sequencing. In general, sample nucleic acids, including nucleic acids flanked by adapters, with or without prior amplification can be subject to sequencing. Sequencing methods include, for example, Sanger sequencing, high-throughput sequencing, pyrosequencing, sequencing-by-synthesis, single-molecule sequencing, nanopore sequencing, semiconductor sequencing, sequencing-by-ligation, sequencing-by-hybridization, Digital Gene Expression (Helicos), Next generation sequencing (NGS), Single Molecule Sequencing by Synthesis (SMSS) (Helicos), enzymatic methyl sequencing (EM-Seq), Tet-assisted pyridine borane sequencing (TAPS), massively-parallel sequencing, Clonal Single Molecule Array (Solexa), shotgun sequencing, Ion Torrent, Oxford Nanopore, Roche Genia, Maxim-Gilbert sequencing, primer walking, and sequencing using PacBio, SOLiD, Ion Torrent, or Nanopore platforms.

In some embodiments, sequencing comprises detecting and/or distinguishing unmodified and modified nucleobases. For example, single-molecule real-time (SMRT) sequencing facilitates direct detection of, e.g., 5-methylcytosine and 5-hydroxymethylcytosine as well as unmodified cytosine. See, e.g., Schatz., Nature Methods. 14(4): 347-348 (2017); and U.S. Pat. No. 9,150,918. Sequencing reactions can be performed in a variety of sample processing units, which may multiple lanes, multiple channels, multiple wells, or other mean of processing multiple sample sets substantially simultaneously. Sample processing unit can also include multiple sample chambers to enable processing of multiple runs simultaneously.

In some embodiments, the sequencing comprises targeted sequencing in which one or more genomic regions of interest are sequenced. In some such embodiments, nucleic acids that do not comprise regions of interest are not sequenced. In some embodiments, levels of certain nucleic acids undergo reliably predictive changes in different conditions, states, or tissue or cell types at genomic regions that are targeted for sequencing. Some embodiments comprise non-targeted sequencing, e.g., all genomic regions of the nucleic acids of the sample or subsample are sequenced, or genomic regions are randomly chosen for sequencing. In some embodiments, detecting nucleic acid sequences of the sample or subsample comprises sequencing nucleic acids that are not enriched for genomic regions of interest, e.g., wherein tissue-specific sequences are obtained in a substantially unbiased manner.

The sequencing reactions can be performed on one or more forms of nucleic acids, such as bacterial nucleic acids, nucleic acids known to be tissue-specific, and other nucleic acids in the sample. The sequencing reactions can also be performed on any nucleic acid fragments present in the sample. In some embodiments, sequence coverage of the genome may be less than 5%, 10%, 15%, 20%, 25%, 30%, 40%, 50%, 60%, 70%, 80%, 90%, 95%, 99%, 99.9% or 100%. In some embodiments, the sequence reactions may provide for sequence coverage of at least 5%, 10%, 15%, 20%, 25%, 30%, 40%, 50%, 60%, 70%, or 80% of the genome. Sequence coverage can be performed on at least 5, 10, 20, 70, 100, 200 or 500 different genes, or at most 5000, 2500, 1000, 500 or 100 different genes.

Simultaneous sequencing reactions may be performed using multiplex sequencing. In some cases, cell-free nucleic acids may be sequenced with at least 1000, 2000, 3000, 4000, 5000, 6000, 7000, 8000, 9000, 10000, 50000, 100,000 sequencing reactions. In other cases cell-free nucleic acids may be sequenced with less than 1000, 2000, 3000, 4000, 5000, 6000, 7000, 8000, 9000, 10000, 50000, 100,000 sequencing reactions. Sequencing reactions may be performed sequentially or simultaneously. Subsequent data analysis may be performed on all or part of the sequencing reactions. In some cases, data analysis may be performed on at least 1000, 2000, 3000, 4000, 5000, 6000, 7000, 8000, 9000, 10000, 50000, 100,000 sequencing reactions. In other cases, data analysis may be performed on less than 1000, 2000, 3000, 4000, 5000, 6000, 7000, 8000, 9000, 10000, 50000, 100,000 sequencing reactions. An exemplary read depth is 1000-50000 reads per locus (base).

### G. Analysis

The present methods can be used to diagnose or classify conditions in a subject and/or tissue or cell types of origin in a sample. In some embodiments, the condition is cancer, precancer, or the presence of a tumor or neoplasm. In some embodiments, the condition is the presence of metastasis. In some embodiments, the condition is the absence of metastasis despite the presence of a cancer, precancer, tumor, or neoplasm. In some embodiments, the condition is characterized (e.g., staging cancer or determining heterogeneity of a cancer), response to treatment of a condition is monitored, or prognosis risk of developing a condition or subsequent course of a condition is determined. The present disclosure can also be useful in determining the efficacy of a particular treatment option. Successful treatment options may decrease the amount of detected DNA sequences associated with a cancer in a subject's blood as there may be fewer cancer cells to shed DNA. In other examples, this may not occur. In another example, certain treatment options may be correlated with genetic profiles of cancers over time. This correlation may be useful in selecting a therapy.

Additionally, if a cancer is observed to be in remission after treatment, the present methods can be used to monitor residual disease or recurrence of disease, particularly metastasis.

The cancers and metastases that may be detected may include cancers of or metastases that have spread to blood, brain, lung, skin, nose, throat, liver, bone, pancreas, bowel, rectal, colon, prostate, thyroid, bladder, head, neck, kidney, mouth, and stomach. The detected cancer, precancer, tumor, neoplasm, or metastasis may be a solid state tumor, heterogeneous tumor, or homogenous tumor. Type and/or stage of cancer can be detected from nucleic acid cell materials comprising genetic variations including mutations, rare mutations, indels, copy number variations, transversions, translocations, recombination, inversion, deletions, aneuploidy, partial aneuploidy, polyploidy, chromosomal instability, chromosomal structure alterations, gene fusions, chromosome fusions, gene truncations, gene amplification, gene duplications, chromosomal lesions, DNA lesions, changes in nucleic acid chemical modifications, changes in epigenetic patterns, and changes in nucleic acid 5-methylcytosine.

Information and data generated by the methods disclosed herein can also be used for characterizing a specific form of cancer. Cancers are often heterogeneous in both composition and staging. The methods disclosed herein may allow characterization of specific sub-types of cancer that may be important in the diagnosis or treatment of that specific sub-type. This information may also provide a subject or practitioner clues regarding the prognosis of a specific type of cancer and allow either a subject or practitioner to adapt treatment options in accord with the progress of the disease. Some cancers can progress to become more aggressive and genetically unstable. Other cancers may remain benign, inactive, or dormant. The system and methods of this disclosure may be useful in determining disease progression.

The present methods can be used to diagnose, prognose, monitor or observe metastasis, precancers, cancers, tumors, or neoplasms. In some embodiments, the methods herein do not involve the diagnosing, prognosing or monitoring a fetus and as such are not directed to non-invasive prenatal testing. In other embodiments, these methodologies may be employed in a pregnant subject to diagnose, prognose, monitor or observe cancers or other diseases in an unborn subject whose DNA and other polynucleotides may co-circulate with maternal molecules.

An exemplary method for detecting the presence or absence of metastasis comprises the following steps:
1. Preparing a sample comprising cell materials (e.g., obtaining blood from a human subject).
2. Subjecting the sample to a bisulfite reagent that converts unmodified cytosine to uracil but does not convert 5-methylcytosine.
3. Partitioning the sample into a plurality of subsamples by contacting the sample with an agent that recognizes a nucleic acid modification, the plurality comprising a first subsample and a second subsample, wherein the first subsample comprises the nucleic acid modification in a greater proportion than the second subsample.
4. Eluting and cleaning nucleic acids of the subsamples and optionally detecting DNA sequences using qPCR or digital PCR.
5. Performing end repair and ligating adapters to DNA prior to or concurrently with amplification of the DNA.
6. Capturing adapter-ligated DNA comprising tissue-specific epigenetic target regions.
7. Re-amplifying the captured DNA and assaying in multiplex on an NGS instrument.
8. Analyzing the NGS data, with the molecular tags of the adapters being used to identify unique molecules.

In some embodiments, determining the levels of captured DNA sequences of the sample facilitates detection of the presence or absence of metastasis and/or identification of appropriate treatments.

### III. Additional Features of Certain Disclosed Methods

### A. Partitioning Cell Materials

Disclosed methods herein comprise detecting cell materials in a sample. In such methods, different forms of nucleic acids (e.g., hypermethylated and hypomethylated DNA), cell debris, and chromatin-associated targets can be physically partitioned based on one or more properties or features of the cell materials. This approach can be used to determine, for example, whether certain DNA sequences are hypermethylated or hypomethylated.

Methylation profiling can involve determining methylation patterns across different regions of the genome. For example, after partitioning molecules based on extent of methylation (e.g., relative number of methylated nucleobases per molecule) and sequencing, the sequences of molecules in the different partitions can be mapped to a reference genome. This can show regions of the genome that, compared with other regions, are more highly methylated or are less highly methylated. In this way, genomic regions, in contrast to individual molecules, may differ in their extent of methylation.

Partitioning nucleic acid molecules in a sample can increase a rare signal, e.g., by enriching rare nucleic acid molecules that are more prevalent in one partition of the sample. For example, a genetic variation present in hypermethylated DNA but less (or not) present in hypomethylated DNA can be more easily detected by partitioning a sample into hypermethylated and hypomethylated nucleic acid molecules. By analyzing multiple partitions of a sample, a multi-dimensional analysis of a single molecule can be performed and hence, greater sensitivity can be achieved. Partitioning may include physically partitioning nucleic acid molecules into partitions or subsamples based on the presence or absence of one or more methylated nucleobases. A sample may be partitioned into partitions or subsamples based on a characteristic that is indicative of differential gene expression or increased shedding at a metastasis site. A sample may be partitioned based on a feature, or combination thereof that provides a difference in signal between a normal state and a diseased state or between a normal tissue and a metastatically invaded tissue during analysis of nucleic acids, e.g., cell free DNA (cfDNA), non-cfDNA, tumor DNA, circulating tumor DNA (ctDNA) and cell free nucleic acids (cfNA).

In some embodiments, hypermethylation and/or hypomethylation variable epigenetic target regions are analyzed to determine whether they show differential methylation characteristic of tumor cells or cells of a type that does not normally contribute to the DNA sample being analyzed (such as cfDNA), and/or particular immune cell types.

In some instances, heterogeneous cell materials in a sample are partitioned into two or more partitions (e.g., at least 3, 4, 5, 6 or 7 partitions). In some embodiments, DNA in each partition is differentially tagged. Tagged partitions can then be pooled together for collective sample prep and/or sequencing. The partitioning-tagging-pooling steps can occur more than once, with each round of partitioning occurring based on a different characteristic (examples provided herein), and tagged using differential tags that are distinguished from other partitions and partitioning means. In other instances, the differentially tagged partitions are separately sequenced.

In some embodiments, sequence reads from differentially tagged and pooled DNA are obtained and analyzed in silico. Tags are used to sort reads from different partitions. Analysis to detect genetic variants can be performed on a partition-by-partition level, as well as whole nucleic acid population level. For example, analysis can include in silico analysis to determine genetic variants, such as CNV, SNV, indel, fusion in nucleic acids in each partition. In some instances, in silico analysis can include determining chromatin structure. For example, coverage of sequence reads can be used to determine nucleosome positioning in chromatin. Higher coverage can correlate with higher nucleosome occupancy in genomic region while lower coverage can correlate with lower nucleosome occupancy or nucleosome depleted region (NDR).

Examples of characteristics of cell materials that can be used for partitioning include but are not limited to sequence length, methylation level, sequence mismatch, association with DNA, and association with lipids that are associated with inner cell membranes. Resulting partitions can include one or more of the following: single-stranded DNA (ssDNA), double-stranded DNA (dsDNA), DNA fragments of varying lengths, unmodified proteins, modified proteins, and RNA. In some embodiments, partitioning based on a cytosine modification (e.g., cytosine methylation) or methylation generally is performed and is optionally combined with at least one additional partitioning step, which may be based on any of the foregoing characteristics or forms of DNA. In some embodiments, a heterogeneous population of nucleic acids is partitioned into nucleic acids with one or more epigenetic modifications and without the one or more epigenetic modifications. Examples of epigenetic modifications include presence or absence of methylation; level of methylation; type of methylation (e.g., 5-methylcytosine versus other types of methylation, such as adenine methylation and/or cytosine hydroxymethylation); and association and level of association with one or more proteins, such as histones. Alternatively or additionally, a heterogeneous population of nucleic acids can be partitioned into nucleic acid molecules associated with nucleosomes and nucleic acid molecules devoid of nucleosomes. Alternatively or additionally, a heterogeneous population of nucleic acids may be partitioned into single-stranded DNA (ssDNA) and double-stranded DNA (dsDNA). Alternatively, or additionally, a heterogeneous population of nucleic acids may be partitioned based on nucleic acid length (e.g., molecules of up to 160 bp and molecules having a length of greater than 160 bp)

The agents used to partition populations of nucleic acids within a sample can be affinity agents, such as antibodies with the desired specificity, natural binding partners or variants thereof (Bock et al., Nat Biotech 28: 1106-1114 (2010); Song et al., Nat Biotech 29: 68-72 (2011)), or artificial peptides selected e.g., by phage display to have specificity to a given target. In some embodiments, the agent used in the partitioning is an agent that recognizes a modified nucleobase. In some embodiments, the modified nucleobase recognized by the agent is a modified cytosine, such as a methylcytosine (e.g., 5-methylcytosine). In some embodiments, the modified nucleobase recognized by the agent is a product of a procedure that affects the first nucleobase in the DNA differently from the second nucleobase in the DNA of the sample. In some embodiments, the modified nucleobase may be a “converted nucleobase,” meaning that its base pairing specificity was changed by a procedure. For example, certain procedures convert unmethylated or unmodified cytosine to dihydrouracil, or more generally, at least one modified or unmodified form of cytosine undergoes deamination, resulting in uracil (considered a modified nucleobase in the context of DNA) or a further modified form of uracil. Examples of partitioning agents include antibodies, such as antibodies that recognize a modified nucleobase, which may be a modified cytosine, such as a methylcytosine (e.g., 5-methylcytosine). In some embodiments, the partitioning agent is an antibody that recognizes a modified cytosine other than 5-methylcytosine, such as 5-carboxylcytosine (5caC). Exemplary partitioning agents include methyl binding domain (MBDs) and methyl binding proteins (MBPs) as described herein, including proteins such as MeCP2, MBD2, and antibodies preferentially binding to 5-methylcytosine. Where an antibody is used to immunoprecipitate methylated DNA, the methylated DNA may be recovered in single-stranded form. In such embodiments, a second strand can be synthesized. Hypermethylated (and optionally intermediately methylated) subsamples may then be contacted with a methylation sensitive nuclease that does not cleave hemi-methylated DNA, such as HpaII, BstUI, or Hin6i. Alternatively or in addition, hypomethylated (and optionally intermediately methylated) subsamples may then be contacted with a methylation dependent nuclease that cleaves hemi-methylated DNA.

Additional, non-limiting examples of partitioning agents are histone binding proteins, and molecules that specifically bind a cell debris marker. In some embodiments, the agent is a histone binding protein that can separate nucleic acids bound to histones from free or unbound nucleic acids. Examples of histone binding proteins that can be used in the methods disclosed herein include RBBP4, RbAp48 and SANT domain peptides. In some embodiments, the agent is a molecule that specifically binds a cell debris marker, e.g., Annexin V or an antibody that specifically binds to an inner membrane lipid, such as phosphatidylserine.

In some embodiments, partitioning can comprise both binary partitioning and partitioning based on degree/level of modifications. For example, methylated fragments can be partitioned by methylated DNA immunoprecipitation (MeDIP), or all methylated fragments can be partitioned from unmethylated fragments using methyl binding domain proteins (e.g., MethylMinder Methylated DNA Enrichment Kit (ThermoFisher Scientific). Subsequently, additional partitioning may involve eluting fragments having different levels of methylation by adjusting the salt concentration in a solution with the methyl binding domain and bound fragments. As salt concentration increases, fragments having greater methylation levels are eluted.

In some instances, the final partitions are enriched in nucleic acids having different extents of modifications (overrepresentative or underrepresentative of modifications). Overrepresentation and underrepresentation can be defined by the number of modifications born by a nucleic acid relative to the median number of modifications per strand in a population. For example, if the median number of 5-methylcytosine residues in nucleic acid in a sample is 2, a nucleic acid including more than two 5-methylcytosine residues is overrepresented in this modification and a nucleic acid with 1 or zero 5-methylcytosine residues is underrepresented. The effect of the affinity separation is to enrich for nucleic acids overrepresented in a modification in a bound phase and for nucleic acids underrepresented in a modification in an unbound phase (i.e. in solution). The nucleic acids in the bound phase can be eluted before subsequent processing.

When using MeDIP or MethylMiner®Methylated DNA Enrichment Kit (ThermoFisher Scientific) various levels of methylation can be partitioned using sequential elutions. For example, a hypomethylated partition (no methylation) can be separated from a methylated partition by contacting the nucleic acid population with the MBD from the kit, which is attached to magnetic beads. The beads are used to separate out the methylated nucleic acids from the non-methylated nucleic acids. Subsequently, one or more elution steps are performed sequentially to elute nucleic acids having different levels of methylation. For example, a first set of methylated nucleic acids can be eluted at a salt concentration of 160 mM or higher, e.g., at least 150 mM, at least 200 mM, 300 mM, 400 mM, 500 mM, 600 mM, 700 mM, 800 mM, 900 mM, 1000 mM, or 2000 mM. After such methylated nucleic acids are eluted, magnetic separation is once again used to separate higher level of methylated nucleic acids from those with lower level of methylation. The elution and magnetic separation steps can be repeated to create various partitions such as a hypomethylated partition (enriched in nucleic acids comprising no methylation), a methylated partition (enriched in nucleic acids comprising low levels of methylation), and a hyper methylated partition (enriched in nucleic acids comprising high levels of methylation).

In some methods, nucleic acids bound to an agent used for affinity separation based partitioning are subjected to a wash step. The wash step washes off nucleic acids weakly bound to the affinity agent. Such nucleic acids can be enriched in nucleic acids having the modification to an extent close to the mean or median (i.e., intermediate between nucleic acids remaining bound to the solid phase and nucleic acids not binding to the solid phase on initial contacting of the sample with the agent).

The affinity separation results in at least two, and sometimes three or more partitions of nucleic acids with different extents of a modification. While the partitions are still separate, the nucleic acids of at least one partition, and usually two or three (or more) partitions are linked to nucleic acid tags, usually provided as components of adapters, with the nucleic acids in different partitions receiving different tags that distinguish members of one partition from another. The tags linked to nucleic acid molecules of the same partition can be the same or different from one another. But if different from one another, the tags may have part of their code in common so as to identify the molecules to which they are attached as being of a particular partition.

For further details regarding portioning nucleic acid samples based on characteristics such as methylation, see WO2018/119452, which is incorporated herein by reference.

In some embodiments, the partitioning comprises contacting the DNA with a methylation sensitive restriction enzyme (MSRE) and/or a methylation dependent restriction enzyme (MDRE). Following the treatment of the DNA with a MSRE or a MDRE, the DNA may be partitioned based on size to generate hypermethylated (longest DNA molecules following MSRE treatment and shortest DNA fragments following MDRE treatment), intermediate (intermediate length DNA molecules following MSRE or MDRE treatment), and hypomethylated (shortest DNA molecules following MSRE treatment and longest DNA fragments following MDRE treatment) subsamples.

In some embodiments, the partitioning is performed by contacting the nucleic acids with a methyl binding domain (“MBD”) of a methyl binding protein (“MBP”). In some such embodiments, the nucleic acids are contacted with an entire MBP. In some embodiments, an MBD binds to 5-methylcytosine (5mC), and an MBP comprises an MBD and is referred to interchangeably herein as a methyl binding protein or a methyl binding domain protein. In some embodiments, MBD is coupled to paramagnetic beads, such as Dynabeads® M-280 Streptavidin via a biotin linker. Partitioning into fractions with different extents of methylation can be performed by eluting fractions by increasing the NaCl concentration.

In some embodiments, bound DNA is eluted by contacting the antibody or MBD with a protease, such as proteinase K. This may be performed instead of or in addition to elution steps using NaCl as discussed herein.

Examples of agents that recognize a modified nucleobase contemplated herein include, but are not limited to:
(a) MeCP2 and MBD2 are proteins that preferentially binds to 5-methyl-cytosine over unmodified cytosine.
(b) RPL26, PRP8 and the DNA mismatch repair protein MHS6 preferentially bind to 5-hydroxymethyl-cytosine over unmodified cytosine.
(c) FOXK1, FOXK2, FOXP1, FOXP4 and FOXI3 preferably bind to 5-formyl-cytosine over unmodified cytosine (Iurlaro et al., Genome Biol. 14: R119 (2013)).
(d) Antibodies specific to one or more methylated or modified nucleobases or conversion products thereof, such as 5mC, 5caC, or DHU.

In general, elution is a function of the number of modifications, such as the number of methylated sites per molecule, with molecules having more methylation eluting under increased salt concentrations. To elute the DNA into distinct populations based on the extent of methylation, one can use a series of elution buffers of increasing NaCl concentration. Salt concentration can range from about 100 nm to about 2500 mM NaCl. In one embodiment, the process results in three (3) partitions. Molecules are contacted with a solution at a first salt concentration and comprising a molecule comprising an agent that recognizes a modified nucleobase, which molecule can be attached to a capture moiety, such as streptavidin. At the first salt concentration a population of molecules will bind to the agent and a population will remain unbound. The unbound population can be separated as a “hypomethylated” population. For example, a first partition enriched in hypomethylated form of DNA is that which remains unbound at a low salt concentration, e.g., 100 mM or 160 mM. A second partition enriched in intermediate methylated DNA is eluted using an intermediate salt concentration, e.g., between 100 mM and 2000 mM concentration. This is also separated from the sample. A third partition enriched in hypermethylated form of DNA is eluted using a high salt concentration, e.g., at least about 2000 mM.

In some embodiments, a monoclonal antibody raised against 5-methylcytidine (5mC) is used to purify methylated DNA. DNA is denatured, e.g., at 95° C. in order to yield single-stranded DNA fragments. Protein G coupled to standard or magnetic beads as well as washes following incubation with the anti-5mC antibody are used to immunoprecipitate DNA bound to the antibody. Such DNA may then be eluted. Partitions may comprise unprecipitated DNA and one or more partitions eluted from the beads. In some embodiments, the partitions of DNA are desalted and concentrated in preparation for enzymatic steps of library preparation.

In some embodiments, the methods comprise preparing a first pool comprising at least a portion of the DNA of the hypomethylated partition. In some embodiments, the methods comprise preparing a second pool comprising at least a portion of the DNA of the hypermethylated partition. In some embodiments, the first pool further comprises a portion of the DNA of the hypermethylated partition. In some embodiments, the second pool further comprises a portion of the DNA of the hypomethylated partition. In some embodiments, the first pool comprises a majority of the DNA of the hypomethylated partition, and optionally and a minority of the DNA of the hypermethylated partition. In some embodiments, the second pool comprises a majority of the DNA of the hypermethylated partition and a minority of the DNA of the hypomethylated partition. In some embodiments involving an intermediately methylated partition, the second pool comprises at least a portion of the DNA of the intermediately methylated partition, e.g., a majority of the DNA of the intermediately methylated partition. In some embodiments, the first pool comprises a majority of the DNA of the hypomethylated partition, and the second pool comprises a majority of the DNA of the hypermethylated partition and a majority of the DNA of the intermediately methylated partition.

In some embodiments, the methods comprise capturing at least a first set of target regions from the first pool, e.g., wherein the first pool is as set forth in any of the embodiments herein. In some embodiments, the first set comprises sequence-variable target regions. In some embodiments, the first set comprises hypomethylation variable target regions and/or fragmentation variable target regions. In some embodiments, the first set comprises sequence-variable target regions and fragmentation variable target regions. In some embodiments, the first set comprises sequence-variable target regions, hypomethylation variable target regions and fragmentation variable target regions. A step of amplifying DNA in the first pool may be performed before this capture step. In some embodiments, capturing the first set of target regions from the first pool comprises contacting the DNA of the first pool with a first set of target-specific probes. In some embodiments, the first set of target-specific probes comprises target-binding probes specific for the sequence-variable target regions. In some embodiments, the first set of target-specific probes comprises target-binding probes specific for the sequence-variable target regions, hypomethylation variable target regions and/or fragmentation variable target regions. In some such embodiments, information obtained using sequence-variable target regions is useful for detecting a primary cancer, precancer, tumor, or neoplasm but is not useful for detecting the presence or absence of a metastasis.

In some embodiments, the methods comprise capturing a second set of target regions or plurality of sets of target regions from the second pool, e.g., wherein the first pool is as set forth in any of the embodiments herein. In some embodiments, the second plurality comprises epigenetic target regions, such as hypermethylation variable target regions and/or fragmentation variable target regions. In some embodiments, the second plurality comprises sequence-variable target regions and epigenetic target regions, such as hypermethylation variable target regions and/or fragmentation variable target regions. A step of amplifying DNA in the second pool may be performed before this capture step. In some embodiments, capturing the second plurality of sets of target regions from the second pool comprises contacting the DNA of the first pool with a second set of target-specific probes, wherein the second set of target-specific probes comprises target-binding probes specific for the sequence-variable target regions and target-binding probes specific for the epigenetic target regions. In some embodiments, the first set of target regions and the second set of target regions are not identical. For example, the first set of target regions may comprise one or more target regions not present in the second set of target regions. Alternatively or in addition, the second set of target regions may comprise one or more target regions not present in the first set of target regions. In some embodiments, at least one hypermethylation variable target region is captured from the second pool but not from the first pool. In some embodiments, a plurality of hypermethylation variable target regions is captured from the second pool but not from the first pool. In some embodiments, the first set of target regions comprises sequence-variable target regions and/or the second set of target regions comprises epigenetic target regions. In some embodiments, the first set of target regions comprises sequence-variable target regions, and fragmentation variable target regions; and the second set of target regions comprises epigenetic target regions, such as hypermethylation variable target regions and fragmentation variable target regions. In some embodiments, the first set of target regions comprises sequence-variable target regions, fragmentation variable target regions, and comprises hypomethylation variable target regions; and the second set of target regions comprises epigenetic target regions, such as hypermethylation variable target regions and fragmentation variable target regions.

In some embodiments, the first pool comprises a majority of the DNA of the hypomethylated partition and a portion of the DNA of the hypermethylated partition (e.g., about half), and the second pool comprises a portion of the DNA of the hypermethylated partition (e.g., about half). In some such embodiments, the first set of target regions comprises sequence-variable target regions and/or the second set of target regions comprises epigenetic target regions. The sequence-variable target regions and/or the epigenetic target regions may be as set forth in any of the embodiments described elsewhere herein.

### B. Enriching/Capturing Epigenetic and Sequence-Variable Target Region Sets

Methods disclosed herein comprise detecting cell materials released from a potential metastasis site and can comprise enriching or capturing DNA comprising epigenetic target regions and/or sequence-variable target regions. In some embodiments, the detecting cell materials released from a potential metastasis site comprises enriching or capturing DNA comprising epigenetic target regions and/or sequence-variable target regions, which facilitates detecting the presence or absence of a metastasis. In some embodiments, methods herein comprise enriching or capturing DNA comprising epigenetic target regions and/or sequence-variable target regions, which facilitates detecting the presence or absence of a primary cancer, precancer, tumor, or neoplasm. In some embodiments, the capturing comprises contacting DNA in the sample or a subsample thereof with probes specific for such target regions. Such enrichment or capture may be performed on any sample or subsample described herein using any suitable approach known in the art.

In some embodiments, the probes specific for the target regions (i.e., target-specific probes) comprise a capture moiety that facilitates the enrichment or capture of the DNA hybridized to the probes. In some embodiments, the capture moiety is biotin. In some such embodiments, streptavidin attached to a solid support, such as magnetic beads, is used to bind to the biotin. Nonspecifically bound DNA that does not comprise a target region is washed away from the captured DNA. In some embodiments, DNA is then dissociated from the probes and eluted from the solid support using salt washes or buffers comprising another DNA denaturing agent. In some embodiments, the probes are also eluted from the solid support by, e.g., disrupting the biotin-streptavidin interaction. In some embodiments, captured DNA is amplified following elution from the solid support. In some such embodiments, DNA comprising adapters is amplified using PCR primers that anneal to the adapters. In some embodiments, captured DNA is amplified while attached to the solid support. In some such embodiments, the amplification comprises use of a PCR primer that anneals to a sequence within an adapter and a PCR primer that anneals to a sequence within a probe annealed to the target region of the DNA.

In some embodiments, the methods herein comprise enriching for or capturing DNA comprising epigenetic and/or sequence-variable target regions. Such regions may be captured from an aliquot, portion, or subsample of a sample (e.g., a sample that has undergone attachment of adapters and amplification), while a step of partitioning the DNA may be performed on a separate aliquot, portion, or subsample of the sample. Enriching for or capturing DNA comprising epigenetic and/or sequence-variable target regions may comprise contacting the DNA with a first or second set of target-specific probes. Such target-specific probes may have any of the features described herein for sets of target-specific probes, including but not limited to in the embodiments set forth herein and the sections relating to probes herein. Capturing may be performed on one or more subsamples prepared during methods disclosed herein. In some embodiments, DNA is captured from a first subsample or a second subsample. In some embodiments, the subsamples are differentially tagged (e.g., as described herein) and then pooled before undergoing capture. Exemplary methods for capturing DNA comprising epigenetic and/or sequence-variable target regions can be found in, e.g., WO 2020/160414, which is hereby incorporated by reference.

The capturing step or steps may be performed using conditions suitable for specific nucleic acid hybridization, which generally depend to some extent on features of the probes such as length, base composition, etc. Those skilled in the art will be familiar with appropriate conditions given general knowledge in the art regarding nucleic acid hybridization.

In some embodiments, methods described herein comprise capturing a plurality of sets of target regions of cfDNA obtained from a subject. The target regions may comprise differences depending on whether they originated from a tumor or from healthy cells of a certain cell type. The capturing step produces a captured set of cfDNA molecules. In some embodiments, cfDNA molecules corresponding to a sequence-variable target region set are captured at a greater capture yield in the captured set of cfDNA molecules than cfDNA molecules corresponding to an epigenetic target region set. In some embodiments, a method described herein comprises contacting cfDNA obtained from a subject with a set of target-specific probes, wherein the set of target-specific probes is configured to capture cfDNA corresponding to the sequence-variable target region set at a greater capture yield than cfDNA corresponding to the epigenetic target region set. For additional discussion of capturing steps, capture yields, and related aspects, see WO2020/160414, which is incorporated herein by reference for all purposes.

It can be beneficial to capture cfDNA corresponding to the sequence-variable target region set at a greater capture yield than cfDNA corresponding to the epigenetic target region set because a greater depth of sequencing may be necessary to analyze the sequence-variable target regions with sufficient confidence or accuracy than may be necessary to analyze the epigenetic target regions. The volume of data needed to determine fragmentation patterns (e.g., to test for perturbation of transcription start sites or CTCF binding sites) or fragment abundance (e.g., in hypermethylated and hypomethylated partitions) is generally less than the volume of data needed to determine the presence or absence of cancer-related sequence mutations. Capturing the target region sets at different yields can facilitate sequencing the target regions to different depths of sequencing in the same sequencing run (e.g., using a pooled mixture and/or in the same sequencing cell). Although copy number variations such as focal amplifications are somatic mutations, they can be detected by sequencing based on read frequency in a manner analogous to approaches for detecting certain epigenetic changes such as changes in methylation.

In some embodiments, the captured DNA is amplified. In various embodiments, the methods further comprise sequencing the captured DNA, e.g., to different degrees of sequencing depth for the epigenetic and sequence-variable target region sets, consistent with the discussion herein. In some embodiments, RNA probes are used. In some embodiments, DNA probes are used. In some embodiments, single stranded probes are used. In some embodiments, double stranded probes are used. In some embodiments, single stranded RNA probes are used. In some embodiments, double stranded DNA probes are used.

In some embodiments, a capturing step is performed with probes for a sequence-variable target region set and probes for an epigenetic target region set in the same vessel at the same time, e.g., the probes for the sequence-variable and epigenetic target region sets and capture probes are in the same composition. This approach provides a relatively streamlined workflow.

In some embodiments, adapters are included in the DNA as described herein. In some embodiments, tags, which may be or include barcodes, are included in the DNA. In some embodiments, such tags are included in adapters. Tags can facilitate identification of the origin of a nucleic acid. For example, barcodes can be used to allow the origin (e.g., subject) whence the DNA came to be identified following pooling of a plurality of samples for parallel sequencing. This may be done concurrently with an amplification procedure, e.g., by providing the barcodes in a 5′ portion of a primer, e.g., as described herein. In some embodiments, adapters and tags/barcodes are provided by the same primer or primer set. For example, the barcode may be located 3′ of the adapter and 5′ of the target-hybridizing portion of the primer. Alternatively, barcodes can be added by other approaches, such as ligation, optionally together with adapters in the same ligation substrate.

Additional details regarding amplification, tags, and barcodes are discussed herein, which can be combined to the extent practicable with any of these embodiments.

### C. Captured DNA; Target Regions

In some embodiments, nucleic acids captured or enriched from a sample or subsample using a method described herein comprise captured DNA. In some embodiments, the captured DNA comprises variations present in healthy cells but not normally present in the sample type, such as a blood sample. In some embodiments, the captured DNA comprises variations present in healthy cells but not normally present at the level observed in the sample, such as a blood sample. In some embodiments, the variations are present in aberrant cells (e.g., hyperplastic, metaplastic, or neoplastic cells).

In some embodiments, a first target region set is captured, comprising at least epigenetic target regions. In some embodiments, the epigenetic target regions captured from a sample or first subsample comprise hypermethylation variable target regions. In some embodiments, the hypermethylation variable target regions are CpG-containing regions that are unmethylated or have low methylation in cfDNA from healthy subjects (e.g., below-average methylation relative to bulk cfDNA). In some embodiments, the hypermethylation variable target regions show type-specific hypermethylation in healthy cfDNA from one or more related cell or tissue types. Without wishing to be bound by any particular theory, the presence of cancer cells, including metastatic cancer cells, may increase the shedding of DNA into the bloodstream (e.g., from the cancer, the surrounding tissue, or the metastatic site). As such, the distribution of tissue of origin of cfDNA may change upon carcinogenesis or upon metastasis. Thus, an increase in the level of hypermethylation variable target regions in the first subsample can be an indicator of the presence (or recurrence, depending on the history of the subject) of cancer. An increase in the level of normally methylated, tissue-specific target regions can be an indicator of the presence of metastasis at the tissue.

In some embodiments, the methods herein comprise capturing a second captured target region set from a sample or second subsample, comprising at least epigenetic target regions. In some embodiments, the second epigenetic target region set comprises hypomethylation variable target regions. In some embodiments, the hypomethylation variable target regions are CpG-containing regions that are methylated or have high methylation in cfDNA from healthy subjects (e.g., above-average methylation relative to bulk cfDNA). Without wishing to be bound by any particular theory, cancer cells and cells surrounding cancer cells, such as those at a metastatic site, may shed more DNA into the bloodstream than healthy cells of the same tissue type. As such, the distribution of tissue of origin of cfDNA may change upon carcinogenesis or metastasis. Thus, an increase in the level of hypomethylation variable target regions in the second subsample can be an indicator of the presence (or recurrence, depending on the history of the subject) of cancer or metastasis.

Additionally, captured target region sets may comprise DNA corresponding to a sequence-variable target region set. The captured sets may be combined to provide a combined captured set.

In some embodiments in which a captured set comprising DNA corresponding to a sequence-variable target region set and the epigenetic target region set includes a combined captured set as discussed herein, the DNA corresponding to the sequence-variable target region set may be present at a greater concentration than the DNA corresponding to the epigenetic target region set, e.g., a 1.1 to 1.2-fold greater concentration, a 1.2- to 1.4-fold greater concentration, a 1.4- to 1.6-fold greater concentration, a 1.6- to 1.8-fold greater concentration, a 1.8- to 2.0-fold greater concentration, a 2.0- to 2.2-fold greater concentration, a 2.2- to 2.4-fold greater concentration a 2.4- to 2.6-fold greater concentration, a 2.6- to 2.8-fold greater concentration, a 2.8- to 3.0-fold greater concentration, a 3.0- to 3.5-fold greater concentration, a 3.5- to 4.0, a 4.0- to 4.5-fold greater concentration, a 4.5- to 5.0-fold greater concentration, a 5.0- to 5.5-fold greater concentration, a 5.5- to 6.0-fold greater concentration, a 6.0- to 6.5-fold greater concentration, a 6.5- to 7.0-fold greater, a 7.0- to 7.5-fold greater concentration, a 7.5- to 8.0-fold greater concentration, an 8.0- to 8.5-fold greater concentration, an 8.5- to 9.0-fold greater concentration, a 9.0- to 9.5-fold greater concentration, 9.5- to 10.0-fold greater concentration, a 10- to 11-fold greater concentration, an 11- to 12-fold greater concentration a 12- to 13-fold greater concentration, a 13- to 14-fold greater concentration, a 14- to 15-fold greater concentration, a 15- to 16-fold greater concentration, a 16- to 17-fold greater concentration, a 17- to 18-fold greater concentration, an 18- to 19-fold greater concentration, a 19- to 20-fold greater concentration, a 20- to 30-fold greater concentration, a 30- to 40-fold greater concentration, a 40- to 50-fold greater concentration, a 50- to 60-fold greater concentration, a 60- to 70-fold greater concentration, a 70- to 80-fold greater concentration, a 80- to 90-fold greater concentration, or a 90- to 100-fold greater concentration. The degree of difference in concentrations accounts for normalization for the footprint sizes of the target regions, as discussed in the definition section.

### 1. Target Regions

In some embodiments, captured DNA comprises epigenetic and/or sequence-variable target regions. In some embodiments, an epigenetic target region set consists of target regions having a type-specific epigenetic variation. In some embodiments, the epigenetic variations, e.g., differential methylation or a fragmentation pattern, are likely to differentiate DNA from one or more related cell or tissue types cells from DNA from other cell or tissue types present in a sample or in a subject. In some embodiments, the epigenetic variations, e.g., differential methylation or a fragmentation pattern, are likely to differentiate DNA from a sample from a healthy subject from DNA from a sample from a subject having a condition or disease state.

In some embodiments, a captured epigenetic target region set captured from a sample or first subsample comprises hypermethylation variable target regions. In some embodiments, the hypermethylation variable target regions are differentially or exclusively hypermethylated in one or more related cell or tissue types. Such hypermethylation variable target regions may be hypermethylated in other cell or tissue types but not to the extent observed in the one or more related cell or tissue types. In some embodiments, the hypermethylation variable target regions show even higher methylation in cfDNA from a diseased cell of the one or more related cell or tissue types.

In some embodiments, a captured epigenetic target region set captured from a sample or subsample comprises hypomethylation variable target regions. In some embodiments, the hypomethylation variable target regions are exclusively hypomethylated in one or more related cell or tissue types. Such hypomethylation variable target regions may be hypomethylated in other cell or tissue types but not to the extent observed in the one or more related cell or tissue types.

Further exemplary hypermethylation variable target regions and hypomethylation variable target regions useful for distinguishing between various cell types have been identified by analyzing DNA obtained from various cell types via whole gnome bisulfite sequencing, as described, e.g., in Scott, C. A., Duryea, J. D., MacKay, H. et al., “Identification of cell type-specific methylation signals in bulk whole genome bisulfite sequencing data,” Genome Biol 21, 156 (2020) (doi.org/10.1186/s13059-020-02065-5). Whole-genome bisulfite sequencing data is available from the Blueprint consortium, available on the internet at dcc.blueprint-epigenome.eu.

In some embodiments, the epigenetic target region set has a footprint of at least 100 kbp, e.g., at least 200 kbp, at least 300 kbp, or at least 400 kbp. In some embodiments, the epigenetic target region set has a footprint in the range of 100-20 Mbp, e.g., 100-200 kbp, 200-300 kbp, 300-400 kbp, 400-500 kbp, 500-600 kbp, 600-700 kbp, 700-800 kbp, 800-900 kbp, 900-1,000 kbp, 1-1.5 Mbp, 1.5-2 Mbp, 2-3 Mbp, 3-4 Mbp, 4-5 Mbp, 5-6 Mbp, 6-7 Mbp, 7-8 Mbp, 8-9 Mbp, 9-10 Mbp, or 10-20 Mbp. In some embodiments, the epigenetic target region set has a footprint of at least 20 Mbp.

In some embodiments, first and second captured target region sets comprise, respectively, DNA corresponding to a sequence-variable target region set and DNA corresponding to the epigenetic target region set, for example, as described in WO 2020/160414. The first and second captured sets may be combined to provide a combined captured set. In some embodiments, the sequence-variable target region set and epigenetic target region set may have any of the features described for such sets in WO 2020/160414, which is incorporated by reference herein in its entirety. In some embodiments, the epigenetic target region set comprises a hypermethylation variable target region set. In some embodiments, the epigenetic target region set comprises a hypomethylation variable target region set. In some embodiments, the epigenetic target region set comprises CTCF binding regions. In some embodiments, the epigenetic target region set comprises fragmentation variable target regions. In some embodiments, the epigenetic target region set comprises transcriptional start sites.

In some embodiments, the sequence-variable target region set comprises a plurality of regions known to undergo somatic mutations in cancer. In some aspects, the sequence-variable target region set targets a plurality of different genes or genomic regions (“panel”) selected such that a determined proportion of subjects having a cancer exhibits a genetic variant or tumor marker in one or more different genes or genomic regions in the panel. The panel may be selected to limit a region for sequencing to a fixed number of base pairs. The panel may be selected to sequence a desired amount of DNA, e.g., by adjusting the affinity and/or amount of the probes as described elsewhere herein. The panel may be further selected to achieve a desired sequence read depth. The panel may be selected to achieve a desired sequence read depth or sequence read coverage for an amount of sequenced base pairs. The panel may be selected to achieve a theoretical sensitivity, a theoretical specificity, and/or a theoretical accuracy for detecting one or more genetic variants in a sample.

Probes for detecting the panel of regions can include those for detecting target regions of interest. Probes can be designed to maximize the likelihood that particular sites (e.g., KRAS codons 12 and 13) can be captured, and may be designed to optimize capture based on analysis of cfDNA coverage and fragment size variation impacted by nucleosome binding patterns and GC sequence composition. Regions used herein can also include regions optimized based on nucleosome positions and GC models.

Examples of listings of target regions of interest may be found in WO 2020/160414, e.g., at Table 4. In some embodiments, a sequence-variable target region set used in the methods of the present disclosure comprises at least a portion of at least 5, at least 10, at least 15, at least 20, at least 25, at least 30, at least 35, at least 40, at least 45, at least 50, at least 55, at least 60, at least 65, or 70 of the genes of Table 3 of WO 2020/160414. In some embodiments, a sequence-variable target region set used in the methods of the present disclosure comprises at least a portion of at least 5, at least 10, at least 15, at least 20, at least 25, at least 30, at least 35, at least 40, at least 45, at least 50, at least 55, at least 60, at least 65, at least 70, or 73 of the genes of Table 4 of WO 2020/160414. Additionally or alternatively, suitable target region sets are available from the literature. For example, Gale et al., PLOS One 13: e0194630 (2018), which is incorporated herein by reference, describes a panel of 35 cancer-related gene targets that can be used as part or all of a sequence-variable target region set. These 35 targets are AKT1, ALK, BRAF, CCND1, CDK2A, CTNNB1, EGFR, ERBB2, ESR1, FGFR1, FGFR2, FGFR3, FOXL2, GATA3, GNA11, GNAQ, GNAS, HRAS, IDH1, IDH2, KIT, KRAS, MED12, MET, MYC, NFE2L2, NRAS, PDGFRA, PIK3CA, PPP2R1A, PTEN, RET, STK11, TP53, and U2AF1.

In some embodiments, the sequence-variable target region set comprises target regions from at least 10, 20, 30, or 35 cancer-related genes, such as the cancer-related genes listed herein and in WO 2020/160414.

In some embodiments, the sequence-variable target region set has a footprint of at least 50 kbp, e.g., at least 100 kbp, at least 200 kbp, at least 300 kbp, or at least 400 kbp. In some embodiments, the sequence-variable target region set has a footprint in the range of 100-2000 kbp, e.g., 100-200 kbp, 200-300 kbp, 300-400 kbp, 400-500 kbp, 500-600 kbp, 600-700 kbp, 700-800 kbp, 800-900 kbp, 900-1,000 kbp, 1-1.5 Mbp or 1.5-2 Mbp. In some embodiments, the sequence-variable target region set has a footprint of at least 2 Mbp.

### D. Capture Moieties

As discussed herein, methods herein may comprise a capture step, in which DNA or other cell materials having certain characteristics are captured and analyzed. DNA capture can involve use of oligonucleotides labeled with a capture moiety, such as target-specific probes labeled with biotin, and a second moiety or binding partner that binds to the capture moiety, such as streptavidin. In some embodiments, a capture moiety and binding partner can have higher and lower capture yields for different sets of probes, such as those used to capture a sequence-variable target region set and an epigenetic target region set, respectively, as discussed elsewhere herein. Methods comprising capture moieties are further described in, for example, U.S. Pat. No. 9,850,523, which is incorporated herein by reference.

Capture moieties include, without limitation, biotin, avidin, streptavidin, a nucleic acid comprising a particular nucleotide sequence, a hapten recognized by an antibody, and magnetically attractable particles. The extraction moiety can be a member of a binding pair, such as biotin/streptavidin or hapten/antibody. In some embodiments, a capture moiety that is attached to an analyte is captured by its binding pair which is attached to an isolatable moiety, such as a magnetically attractable particle or a large particle that can be sedimented through centrifugation. The capture moiety can be any type of molecule that allows affinity separation of nucleic acids bearing the capture moiety from nucleic acids lacking the capture moiety. Exemplary capture moieties are biotin which allows affinity separation by binding to streptavidin linked or linkable to a solid phase or an oligonucleotide, which allows affinity separation through binding to a complementary oligonucleotide linked or linkable to a solid phase.

### E. Procedures That Affect a First Nucleobase in the DNA Differently From a Second Nucleobase in the DNA

In some embodiments, methods disclosed herein comprise a step of subjecting DNA to a procedure that affects a first nucleobase in the DNA differently from a second nucleobase in the DNA, wherein the first nucleobase is a modified or unmodified nucleobase, the second nucleobase is a modified or unmodified nucleobase different from the first nucleobase, and the first nucleobase and the second nucleobase have the same base pairing specificity. In some embodiments, the procedure chemically converts the first or second nucleobase such that the base pairing specificity of the converted nucleobase is altered. In some embodiments, if the first nucleobase is a modified or unmodified adenine, then the second nucleobase is a modified or unmodified adenine; if the first nucleobase is a modified or unmodified cytosine, then the second nucleobase is a modified or unmodified cytosine; if the first nucleobase is a modified or unmodified guanine, then the second nucleobase is a modified or unmodified guanine; and if the first nucleobase is a modified or unmodified thymine, then the second nucleobase is a modified or unmodified thymine (where modified and unmodified uracil are encompassed within modified thymine for the purpose of this step).

In some embodiments, the first nucleobase is a modified or unmodified cytosine, then the second nucleobase is a modified or unmodified cytosine. For example, first nucleobase may comprise unmodified cytosine (C) and the second nucleobase may comprise one or more of 5-methylcytosine (5mC) and 5-hydroxymethylcytosine (5hmC). Alternatively, the second nucleobase may comprise C and the first nucleobase may comprise one or more of 5mC and 5hmC. Other combinations are also possible, such as where one of the first and second nucleobases comprises 5mC and the other comprises 5hmC.

In some embodiments, the procedure that affects a first nucleobase in the DNA differently from a second nucleobase in the DNA comprises bisulfite conversion. Performing bisulfite conversion can facilitate identifying positions containing 5mC or 5hmC using the sequence reads. For an exemplary description of bisulfite conversion, see, e.g., Moss et al., Nat Commun. 2018; 9: 5068.

In some embodiments, the procedure that affects a first nucleobase in the DNA differently from a second nucleobase in the DNA comprises oxidative bisulfite (Ox-BS) conversion. For an exemplary description of oxidative bisulfite conversion, see, e.g., Booth et al., Science 2012; 336: 934-937.

In some embodiments, the procedure that affects a first nucleobase in the DNA differently from a second nucleobase in the DNA comprises Tet-assisted bisulfite (TAB) conversion. For example, as described in Yu et al., Cell 2012; 149: 1368-80, β-glucosyl transferase can be used to protect 5hmC (forming 5-glucosylhydroxymethylcytosine (5ghmC)), then a TET protein such as mTet1 can be used to convert 5mC to 5caC, and then bisulfite treatment can be used to convert C and 5caC to U while 5ghmC remains unaffected.

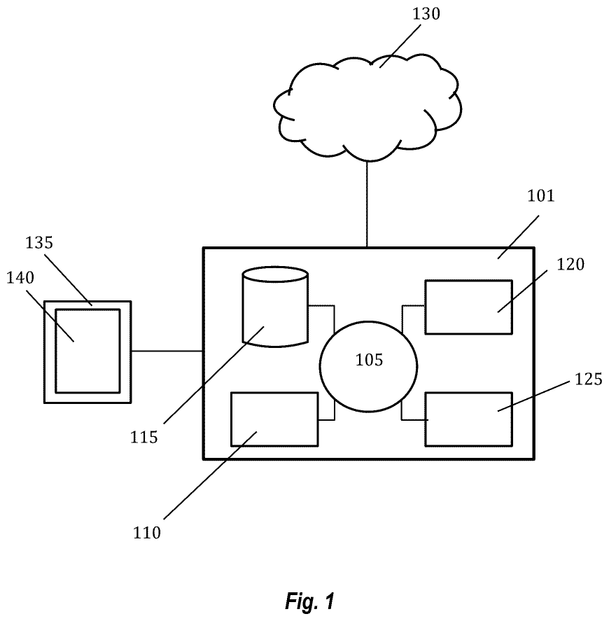

In some embodiments, the procedure that affects a first nucleobase in the DNA differently from a second nucleobase in the DNA comprises Tet-assisted conversion with a substituted borane reducing agent, optionally wherein the substituted borane reducing agent is 2-picoline borane, borane pyridine, tert-butylamine borane, or ammonia borane. See, e.g., Liu et al., Nature Biotechnology 2019; 37:424-429 (e.g., at Supplementary FIG. 1 and Supplementary Note 7). In some embodiments, protection of 5hmC (e.g., using βGT) can be combined with Tet-assisted conversion with a substituted borane reducing agent. Performing TAPSβ conversion can facilitate distinguishing positions containing unmodified C or 5hmC on the one hand from positions containing 5mC using the sequence reads.

In some embodiments, the procedure that affects a first nucleobase in the DNA differently from a second nucleobase in the DNA comprises APOBEC-coupled epigenetic (ACE) conversion. For an exemplary description of ACE conversion, see, e.g., Schutsky et al., Nature Biotechnology 2018; 36: 1083-1090.

Techniques comprising methylated DNA immunoprecipitation (MeDIP) can be used to separate DNA containing modified bases such as 5mC, methylA, 5caC (which may be generated by oxidation of 5mC or 5hmC with Tet2, e.g., before enzymatic conversion of unmodified C to U, e.g., using a deaminase such as APOBEC3A), or dihydrouracil from other DNA. See, e.g., Kumar et al., Frontiers Genet. 2018; 9: 640; Greer et al., Cell 2015; 161: 868-878. An antibody specific for mA is described in Sun et al., Bioessays 2015; 37: 1155-62. Antibodies for various modified nucleobases, such as 5mC, 5caC, and forms of thymine/uracil including dihydrouracil or halogenated forms such as 5-bromouracil, are commercially available. Various modified bases can also be detected based on alterations in their base pairing specificity. For example, hypoxanthine is a modified form of adenine that can result from deamination and is read in sequencing as a G. See, e.g., U.S. Pat. No. 8,486,630; Brown, Genomes, 2^{nd} Ed., John Wiley & Sons, Inc., New York, N.Y., 2002, chapter 14, “Mutation, Repair, and Recombination.”

### F. Computer Systems

Methods of the present disclosure can be implemented using, or with the aid of, computer systems. For example, such methods may comprise detecting a presence or level of at least one cell material released from a potential metastasis site in a sample from a subject. Such methods may further comprise detecting whether the presence or level of the at least one cell material released from the potential metastasis site has changed relative to the presence or level of the at least on cell material in a previous sample from the subject. In another example, methods implemented using, or with the aid of, computer systems may comprise detecting a level of at least one cell material released from a potential metastasis site in a sample for a subject relative to the level of a comparator cell material. Such methods may also comprise subjecting the sample to a procedure that affects a first nucleobase differently than a second nucleobase, partitioning the sample into a plurality of subsamples, capturing DNA comprising epigenetic or sequence-variable target regions, and/or other procedures.

FIG. 1 shows a computer system 101 that is programmed or otherwise configured to implement the methods of the present disclosure. The computer system 101 can regulate various aspects sample preparation, sequencing, and/or analysis. In some examples, the computer system 101 is configured to perform sample preparation and sample analysis, including nucleic acid sequencing, e.g., according to any of the methods disclosed herein.

The computer system 101 includes a central processing unit (CPU, also “processor” and “computer processor” herein) 105, which can be a single core or multi core processor, or a plurality of processors for parallel processing. The computer system 101 also includes memory or memory location 110 (e.g., random-access memory, read-only memory, flash memory), electronic storage unit 115 (e.g., hard disk), communication interface 120 (e.g., network adapter) for communicating with one or more other systems, and peripheral devices 125, such as cache, other memory, data storage, and/or electronic display adapters. The memory 110, storage unit 115, interface 120, and peripheral devices 125 are in communication with the CPU 105 through a communication network or bus (solid lines), such as a motherboard. The storage unit 115 can be a data storage unit (or data repository) for storing data. The computer system 101 can be operatively coupled to a computer network 130 with the aid of the communication interface 120. The computer network 130 can be the Internet, an internet and/or extranet, or an intranet and/or extranet that is in communication with the Internet. The computer network 130 in some cases is a telecommunication and/or data network. The computer network 130 can include one or more computer servers, which can enable distributed computing, such as cloud computing. The computer network 130, in some cases with the aid of the computer system 101, can implement a peer-to-peer network, which may enable devices coupled to the computer system 101 to behave as a client or a server.

The CPU 105 can execute a sequence of machine-readable instructions, which can be embodied in a program or software. The instructions may be stored in a memory location, such as the memory 110. Examples of operations performed by the CPU 105 can include fetch, decode, execute, and writeback.

The storage unit 115 can store files, such as drivers, libraries, and saved programs. The storage unit 115 can store programs generated by users and recorded sessions, as well as output(s) associated with the programs. The storage unit 115 can store user data, e.g., user preferences and user programs. The computer system 101 in some cases can include one or more additional data storage units that are external to the computer system 101, such as located on a remote server that is in communication with the computer system 101 through an intranet or the Internet. Data may be transferred from one location to another using, for example, a communication network or physical data transfer (e.g., using a hard drive, thumb drive, or other data storage mechanism).

The computer system 101 can communicate with one or more remote computer systems through the network 130. For embodiment, the computer system 101 can communicate with a remote computer system of a user (e.g., operator). Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 401 via the network 130.

Methods as described herein can be implemented by way of machine (e.g., computer processor) executable code stored on an electronic storage location of the computer system 101, such as, for example, on the memory 110 or electronic storage unit 115. The machine executable or machine-readable code can be provided in the form of software. During use, the code can be executed by the processor 105. In some cases, the code can be retrieved from the storage unit 115 and stored on the memory 110 for ready access by the processor 105. In some situations, the electronic storage unit 115 can be precluded, and machine-executable instructions are stored on memory 110.

In an aspect, the present disclosure provides a non-transitory computer-readable medium comprising computer-executable instructions which, when executed by at least one electronic processor, perform at least a portion of a method comprising: collecting a sample from a subject; detecting a presence or level of at least one cell material released from a potential metastasis site, wherein the level is optionally relative to a comparator cell material; detecting whether the presence or level of the at least one cell material released from the potential metastasis site has changed relative to the presence of level of the at least one cell material in a previous sample from the subject; and detecting the presence or absence of a metastasis based at least in part on the presence, level, or relative level of the at least one cell material released from the potential metastasis site. In some embodiments, the method performed upon execution further comprises additional features as described herein, such as ligating adapters to and amplifying DNA present in the sample; capturing a plurality of sets of target regions from the DNA, wherein the plurality of target region sets comprises a sequence-variable target region set and an epigenetic target region set, whereby captured DNA, also referred to as a captured set of DNA molecules is produced; sequencing the captured DNA molecules, wherein the captured DNA molecules of the sequence-variable target region set are sequenced to a greater depth of sequencing than the captured DNA molecules of the epigenetic target region set; obtaining a plurality of sequence reads generated by a nucleic acid sequencer from sequencing the captured DNA molecules; mapping the plurality of sequence reads to one or more reference sequences to generate mapped sequence reads; and processing the mapped sequence reads corresponding to the sequence-variable target region set and to the epigenetic target region set to determine the level, tissue of origin, and/or disease state of the DNA.

The code can be pre-compiled and configured for use with a machine have a processer adapted to execute the code or can be compiled during runtime. The code can be supplied in a programming language that can be selected to enable the code to execute in a pre-compiled or as-compiled fashion.

Aspects of the systems and methods provided herein, such as the computer system 101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming.

All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as those used across physical interfaces between local devices, through wired and optical landline networks, and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

Hence, a machine-readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards, paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 101 can include or be in communication with an electronic display that comprises a user interface (UI) for providing, for example, one or more results of sample analysis. Examples of UIs include, without limitation, a graphical user interface (GUI) and web-based user interface.

Additional details relating to computer systems and networks, databases, and computer program products are also provided in, for example, Peterson, Computer Networks: A Systems Approach, Morgan Kaufmann, 5^{th} Ed. (2011), Kurose, Computer Networking: A Top-Down Approach, Pearson, 7^{th} Ed. (2016), Elmasri, Fundamentals of Database Systems, Addison Wesley, 6^{th} Ed. (2010), Coronel, Database Systems: Design, Implementation, & Management, Cengage Learning, 11^{th} Ed. (2014), Tucker, Programming Languages, McGraw-Hill Science/Engineering/Math, 2^{nd} Ed. (2006), and Rhoton, Cloud Computing Architected: Solution Design Handbook, Recursive Press (2011), each of which is hereby incorporated by reference in its entirety.

### G. Applications

### 1. Cancer and Other Diseases

The present methods can be used to diagnose the presence of conditions, particularly metastasis and cancer, in a subject, to characterize conditions (e.g., staging cancer or determining heterogeneity of a cancer), monitor response to treatment of a condition, effect prognosis risk of developing a condition or subsequent course of a condition. The present disclosure can also be useful in determining the efficacy of a particular treatment option. Successful treatment options may increase the amount of primary cancer associated enriched DNA sequences detected in the subject's blood if the treatment is successful as more cancers may die and shed DNA. In other examples, this may not occur. Successful treatment options may alter the presence or level of at least one cell material released from a potential metastasis site. For example, treatments may initially increase and then decrease the level of such materials (e.g., if the metastasis is partially or completely eliminated). In another example, successful treatment options may simply decrease the level (e.g., if the growth of metastasis is arrested or if the metastasis is rapidly eliminated). In another example, certain treatment options may be correlated with genetic profiles of cancers over time. This correlation may be useful in selecting a therapy.

In some embodiments, the present methods are used for screening for a cancer, such as a metastasis, or in a method for screening cancer, such as in a method of detecting the presence or absence of a metastasis. For example, the sample can be a sample from a subject who has or has not been previously diagnosed with cancer. In some embodiments, one or more, such as 1, 2, 3, 4, 5, 6, 7, 8, 9, 10, or more samples are collected from a subject as described herein, such as before and/or after the subject is diagnosed with a cancer. In some embodiments, the subject may or may not have cancer. In some embodiments, the subject may or may not have an early-stage cancer. In some embodiments, the subject has one or more risk factors for cancer, such as tobacco use (e.g., smoking), being overweight or obese, having a high body mass index (BMI), being of advanced age, poor nutrition, high alcohol consumption, or a family history of cancer.

In some embodiments, the subject has used tobacco, e.g., for at least 1, 5, 10, or 15 years. In some embodiments, the subject has a high BMI, e.g., a BMI of 25 or greater, 26 or greater, 27 or greater, 28 or greater, 29 or greater, or 30 or greater. In some embodiments, the subject is at least 40, 45, 50, 55, 60, 65, 70, 75, or 80 years old. In some embodiments, the subject has poor nutrition, e.g., high consumption of one or more of red meat and/or processed meat, trans fat, saturated fat, and refined sugars, and/or low consumption of fruits and vegetables, complex carbohydrates, and/or unsaturated fats. High and low consumption can be defined, e.g., as exceeding or falling below, respectively, recommendations in Dietary Guidelines for Americans 2020-2025, available at www.dietaryguidelines.gov/sites/default/files/2021-03/Dietary_Guidelines_for_Americans-2020-2025.pdf. In some embodiments, the subject has high alcohol consumption, e.g., at least three, four, or five drinks per day on average (where a drink is about one ounce or 30 mL of 80-proof hard liquor or the equivalent). In some embodiments, the subject has a family history of cancer, e.g., at least one, two, or three blood relatives were previously diagnosed with cancer. In some embodiments, the relatives are at least third-degree relatives (e.g., great-grandparent, great aunt or uncle, first cousin), at least second-degree relatives (e.g., grandparent, aunt or uncle, or half-sibling), or first-degree relatives (e.g., parent or full sibling).

Additionally, if a cancer is observed to be in remission after treatment, the present methods can be used to monitor residual disease or recurrence or metastasis of disease.

In some embodiments, the methods and systems disclosed herein may be used to identify customized or targeted therapies to treat a given disease or condition in patients, such as specific treatment of a metastasis site. Typically, the disease under consideration is a type of cancer or metastasis of the cancer. Non-limiting examples of such cancers include biliary tract cancer, bladder cancer, transitional cell carcinoma, urothelial carcinoma, brain cancer, gliomas, astrocytomas, breast carcinoma, metaplastic carcinoma, cervical cancer, cervical squamous cell carcinoma, rectal cancer, colorectal carcinoma, colon cancer, hereditary nonpolyposis colorectal cancer, colorectal adenocarcinomas, gastrointestinal stromal tumors (GISTs), endometrial carcinoma, endometrial stromal sarcomas, esophageal cancer, esophageal squamous cell carcinoma, esophageal adenocarcinoma, ocular melanoma, uveal melanoma, gallbladder carcinomas, gallbladder adenocarcinoma, renal cell carcinoma, clear cell renal cell carcinoma, transitional cell carcinoma, urothelial carcinomas, Wilms tumor, leukemia, acute lymphocytic leukemia (ALL), acute myeloid leukemia (AML), chronic lymphocytic leukemia (CLL), chronic myeloid leukemia (CML), chronic myelomonocytic leukemia (CMML), liver cancer, liver carcinoma, hepatoma, hepatocellular carcinoma, cholangiocarcinoma, hepatoblastoma, Lung cancer, non-small cell lung cancer (NSCLC), mesothelioma, B-cell lymphomas, non-Hodgkin lymphoma, diffuse large B-cell lymphoma, Mantle cell lymphoma, T cell lymphomas, non-Hodgkin lymphoma, precursor T-lymphoblastic lymphoma/leukemia, peripheral T cell lymphomas, multiple myeloma, nasopharyngeal carcinoma (NPC), neuroblastoma, oropharyngeal cancer, oral cavity squamous cell carcinomas, osteosarcoma, ovarian carcinoma, pancreatic cancer, pancreatic ductal adenocarcinoma, pseudopapillary neoplasms, acinar cell carcinomas. Prostate cancer, prostate adenocarcinoma, skin cancer, melanoma, malignant melanoma, cutaneous melanoma, small intestine carcinomas, stomach cancer, gastric carcinoma, gastrointestinal stromal tumor (GIST), uterine cancer, or uterine sarcoma. Type and/or stage of cancer can be detected from genetic variations including mutations, rare mutations, indels, copy number variations, transversions, translocations, inversion, deletions, aneuploidy, partial aneuploidy, polyploidy, chromosomal instability, chromosomal structure alterations, gene fusions, chromosome fusions, gene truncations, gene amplification, gene duplications, chromosomal lesions, DNA lesions, abnormal changes in nucleic acid chemical modifications, abnormal changes in epigenetic patterns, and abnormal changes in nucleic acid 5-methylcytosine.

Methods herein can also be used for characterizing a specific form of cancer. Cancers are often heterogeneous in both composition and staging. Characterization of specific sub-types of cancer may be important in the diagnosis or treatment of that specific sub-type. This information may also provide a subject or practitioner clues regarding the prognosis of a specific type of cancer and allow either a subject or practitioner to adapt treatment options in accord with the progress of the disease. Some cancers can progress to become more aggressive and genetically unstable. Other cancers may remain benign, inactive or dormant. The system and methods of this disclosure may be useful in determining disease progression.

Further, the methods of the disclosure may be used to characterize the heterogeneity of an abnormal condition in a subject. In some embodiments, an abnormal condition is cancer. In some embodiments, the abnormal condition may be one resulting in a heterogeneous genomic population. In the example of cancer, some tumors are known to comprise tumor cells in different stages of the cancer. In other examples, heterogeneity may comprise multiple foci of disease, where one or more foci are the result of metastases that have spread from a primary site. The tissue(s) of origin can be useful for identifying organs affected by the cancer, including the primary cancer and/or metastatic tumors.

The present methods can also be used to quantify levels of different cell types, such as immune cell types, including rare immune cell types, such as activated lymphocytes and myeloid cells at particular stages of differentiation. Such quantification can be based on the numbers of molecules corresponding to a given cell type in a sample. Sequence information obtained in the present methods may comprise sequence reads of the nucleic acids generated by a nucleic acid sequencer. In some embodiments, the nucleic acid sequencer performs pyrosequencing, single-molecule sequencing, nanopore sequencing, semiconductor sequencing, sequencing-by-synthesis, 5-letter sequencing, 6-letter sequencing, sequencing-by-ligation or sequencing-by-hybridization on the nucleic acids to generate sequencing reads. In some embodiments, the method further comprises grouping the sequence reads into families of sequence reads, each family comprising sequence reads generated from a nucleic acid in the sample. In some embodiments, the methods comprise determining the likelihood that the subject from which the sample was obtained has cancer or precancer, or has a metastasis, that is related to changes in proportions of types of immune cells.

The present methods can be used to diagnose, prognose, monitor or observe cancers, or other diseases. In some embodiments, the methods herein do not involve the diagnosing, prognosing or monitoring a fetus and as such are not directed to non-invasive prenatal testing. In other embodiments, these methodologies may be employed in a pregnant subject to diagnose, prognose, monitor or observe cancers or other diseases in an unborn subject whose DNA and other polynucleotides may co-circulate with maternal molecules.

In some embodiments, a method described herein comprises detecting a presence or absence of a nucleic acid originating or derived from a tumor cell at a preselected timepoint following a previous cancer treatment of a subject previously diagnosed with cancer. The method may further comprise determining a cancer recurrence score that is indicative of the presence or levels of DNA originating or derived from the tumor cell for the subject. Cell materials released from a potential metastasis site may be detected simultaneously at the preselected timepoint or at a different time, e.g., using a different sample.

Where a cancer recurrence score is determined, it may further be used to determine a cancer recurrence status. The cancer recurrence status may be at risk for cancer recurrence, e.g., when the cancer recurrence score is above a predetermined threshold. The cancer recurrence status may be at low or lower risk for cancer recurrence, e.g., when the cancer recurrence score is above a predetermined threshold. In particular embodiments, a cancer recurrence score equal to the predetermined threshold may result in a cancer recurrence status of either at risk for cancer recurrence or at low or lower risk for cancer recurrence.

In some embodiments, a cancer recurrence score is compared with a predetermined cancer recurrence threshold, and the subject is classified as a candidate for a subsequent cancer treatment when the cancer recurrence score is above the cancer recurrence threshold or not a candidate for therapy when the cancer recurrence score is below the cancer recurrence threshold. In particular embodiments, a cancer recurrence score equal to the cancer recurrence threshold may result in classification as either a candidate for a subsequent cancer treatment or not a candidate for therapy.

The methods discussed herein may further comprise any compatible feature or features set forth elsewhere herein, including in the section regarding methods of determining a risk of cancer recurrence in a subject and/or classifying a subject as being a candidate for a subsequent cancer treatment.

### 2. Methods of Determining a Risk of Cancer Recurrence or Presence or Absence of Metastasis in a Subject and/or Classifying a Subject as Being a Candidate for a Subsequent Cancer Treatment

In some embodiments, a method provided herein is or comprises a method of determining a risk of cancer recurrence in a subject. In some embodiments, a method provided herein is or comprises a method of detecting the presence of absence of a metastasis in a subject. In some embodiments, a method provided herein is or comprises a method of classifying a subject as being a candidate for a subsequent cancer treatment.

Any of such methods may comprise collecting a sample from the subject diagnosed with the cancer at one or more preselected timepoints following one or more previous cancer treatments to the subject. The subject may be any of the subjects described herein. The sample may comprise chromatin, cfDNA, or other cell materials.

Any of such methods may comprise detecting cell materials released from a potential metastatic site. Any of such methods may comprise sequencing DNA molecules, whereby a set of sequence information is produced. Any of such methods may comprise detecting a presence or absence of DNA originating or derived from a tumor cell at a preselected timepoint using the set of sequence information. The detection of the presence or absence of DNA originating or derived from a tumor cell may be performed according to any of the embodiments thereof described elsewhere herein.

In any of such methods, the previous cancer treatment may comprise surgery, administration of a therapeutic composition, and/or chemotherapy.

Methods of determining a risk of cancer recurrence in a subject may comprise determining a cancer recurrence score that is indicative of the presence or absence, or amount, of genomic regions of interest and target regions originating or derived from the tumor cell for the subject. The cancer recurrence score may further be used to determine a cancer recurrence status. The cancer recurrence status may be at risk for cancer recurrence, e.g., when the cancer recurrence score is above a predetermined threshold. The cancer recurrence status may be at low or lower risk for cancer recurrence, e.g., when the cancer recurrence score is above a predetermined threshold. In particular embodiments, a cancer recurrence score equal to the predetermined threshold may result in a cancer recurrence status of either at risk for cancer recurrence or at low or lower risk for cancer recurrence.

Methods of detecting the presence or absence of metastasis in a subject may comprise comparing the presence or level of a tissue-specific cell material to the presence or level of the tissue-specific cell material obtained from the subject at a different time, a reference level of the tissue-specific cell material, or to a comparator cell material. Methods herein may comprise additional steps to determine whether a metastasis is present.

Methods of classifying a subject as being a candidate for a subsequent cancer treatment may comprise comparing the cancer recurrence score of the subject with a predetermined cancer recurrence threshold, thereby classifying the subject as a candidate for the subsequent cancer treatment when the cancer recurrence score is above the cancer recurrence threshold or not a candidate for therapy when the cancer recurrence score is below the cancer recurrence threshold. In particular embodiments, a cancer recurrence score equal to the cancer recurrence threshold may result in classification as either a candidate for a subsequent cancer treatment or not a candidate for therapy. In some embodiments, the subsequent cancer treatment comprises chemotherapy or administration of a therapeutic composition.

Any of such methods may comprise determining a disease-free survival (DFS) period for the subject based on the cancer recurrence score; for example, the DFS period may be 1 year, 2 years, 3, years, 4 years, 5 years, or 10 years.

In some embodiments, the set of sequence information comprises sequence-variable target region sequences and determining the cancer recurrence score may comprise determining at least a first subscore indicative of the levels of particular cell types, SNVs, insertions/deletions, CNVs and/or fusions present in sequence-variable target region sequences.

In some embodiments, a number of mutations in the sequence-variable target regions chosen from 1, 2, 3, 4, or 5 is sufficient for the first subscore to result in a cancer recurrence score classified as positive for cancer recurrence. In some embodiments, the number of mutations is chosen from 1, 2, or 3.

In some embodiments, epigenetic target region sequences are obtained, and determining the cancer recurrence score comprises determining a subscore indicative of the amount of molecules (obtained from the epigenetic target region sequences) that represent an epigenetic state different from DNA found in a corresponding sample from a healthy subject, and/or DNA found in a tissue sample from a healthy subject where the tissue sample is of the same type of tissue as was obtained from the subject). These abnormal molecules (i.e., molecules with an epigenetic state different from DNA found in a corresponding sample from a healthy subject) may be consistent with epigenetic changes associated with cancer (such as with a metastasis), e.g., methylation of hypermethylation variable target regions and/or perturbed fragmentation of fragmentation variable target regions, where “perturbed” means different from DNA found in a corresponding sample from a healthy subject.

In some embodiments, a proportion of molecules corresponding to the hypermethylation variable target region set and/or fragmentation variable target region set that indicate hypermethylation in the hypermethylation variable target region set and/or abnormal fragmentation in the fragmentation variable target region set greater than or equal to a value in the range of 0.001%-10% is sufficient for the subscore to be classified as positive for cancer recurrence. The range may be 0.001%-1%, 0.005%-1%, 0.01%-5%, 0.01%-2%, or 0.01%-1%.

In some embodiments, any of such methods may comprise determining a fraction of tumor DNA from the fraction of molecules in the set of sequence information that indicate one or more features indicative of origination from a tumor cell. This may be done for molecules corresponding to some or all of the target regions, e.g., including one or both of hypermethylation variable target regions, hypomethylation variable target regions, and fragmentation variable target regions (hypermethylation of a hypermethylation variable target region and/or abnormal fragmentation of a fragmentation variable target region may be considered indicative of origination from a tumor cell). This may be done for molecules corresponding to sequence variable target regions, e.g., molecules comprising alterations consistent with cancer, such as SNVs, indels, CNVs, and/or fusions. The fraction of tumor DNA may be determined based on a combination of molecules corresponding to epigenetic target regions and molecules corresponding to sequence variable target regions.

Determination of a cancer recurrence score may be based at least in part on the fraction of tumor DNA, wherein a fraction of tumor DNA greater than a threshold in the range of 10⁻¹¹ to 1 or 10⁻¹⁰ to 1 is sufficient for the cancer recurrence score to be classified as positive for cancer recurrence. In some embodiments, a fraction of tumor DNA greater than or equal to a threshold in the range of 10⁻¹⁰ to 10⁻⁹, 10⁻⁹ to 10⁻⁸, 10⁻⁸ to 10⁻⁷, 10⁻⁷ to 10⁻⁶, 10⁻⁶ to 10⁻⁵, 10⁻⁵ to 10⁻⁴, 10⁻⁴ to 10⁻³, 10⁻³ to 10⁻², or 10⁻² to 10⁻¹ is sufficient for the cancer recurrence score to be classified as positive for cancer recurrence. In some embodiments, the fraction of tumor DNA greater than a threshold of at least 10⁻⁷ is sufficient for the cancer recurrence score to be classified as positive for cancer recurrence. A determination that a fraction of tumor DNA is greater than a threshold, such as a threshold corresponding to any of the foregoing embodiments, may be made based on a cumulative probability. For example, the sample was considered positive if the cumulative probability that the tumor fraction was greater than a threshold in any of the foregoing ranges exceeds a probability threshold of at least 0.5, 0.75, 0.9, 0.95, 0.98, 0.99, 0.995, or 0.999. In some embodiments, the probability threshold is at least 0.95, such as 0.99.

In some embodiments, the set of sequence information comprises sequence-variable target region sequences and epigenetic target region sequences, and determining the cancer recurrence score comprises determining a subscore indicative of the amount of SNVs, insertions/deletions, CNVs and/or fusions present in sequence-variable target region sequences and a subscore indicative of the amount of abnormal molecules in epigenetic target region sequences, and combining the subscores to provide the cancer recurrence score. Where the subscores are combined, they may be combined by applying a threshold to each subscore independently in sequence-variable target regions, respectively, and greater than a predetermined fraction of abnormal molecules (i.e., molecules with an epigenetic state different from the DNA found in a corresponding sample from a healthy subject; e.g., tumor) in epigenetic target regions), or training a machine learning classifier to determine status based on a plurality of positive and negative training samples.

In some embodiments, a value for the combined score in the range of −4 to 2 or −3 to 1 is sufficient for the cancer recurrence score to be classified as positive for cancer recurrence.

In any embodiment where a cancer recurrence score is classified as positive for cancer recurrence, the cancer recurrence status of the subject may be at risk for cancer recurrence and/or the subject may be classified as a candidate for a subsequent cancer treatment. In some embodiments, the cancer is any one of the types of cancer described elsewhere herein.

### 3. Therapies and Related Administration

In certain embodiments, the methods disclosed herein relate to identifying and administering therapies, such as customized therapies to patients. In some embodiments, the patient or subject has a given disease, disorder or condition, e.g., any of the cancers or other conditions described elsewhere herein. Essentially any cancer therapy (e.g., surgical therapy, radiation therapy, chemotherapy, immunotherapy, and/or the like) may be included as part of these methods. In certain embodiments, the therapy administered to a subject comprises at least one chemotherapy drug. In some embodiments, the chemotherapy drug may comprise alkylating agents (for example, but not limited to, Chlorambucil, Cyclophosphamide, Cisplatin and Carboplatin), nitrosoureas (for example, but not limited to, Carmustine and Lomustine), anti-metabolites (for example, but not limited to, Fluorauracil, Methotrexate and Fludarabine), plant alkaloids and natural products (for example, but not limited to, Vincristine, Paclitaxel and Topotecan), anti-tumor antibiotics (for example, but not limited to, Bleomycin, Doxorubicin and Mitoxantrone), hormonal agents (for example, but not limited to, Prednisone, Dexamethasone, Tamoxifen and Leuprolide) and biological response modifiers (for example, but not limited to, Herceptin and Avastin, Erbitux and Rituxan). In some embodiments, the chemotherapy administered to a subject may comprise FOLFOX or FOLFIRI. In certain embodiments, a therapy may be administered to a subject that comprises at least one PARP inhibitor. In certain embodiments, the PARP inhibitor may include OLAPARIB, TALAZOPARIB, RUCAPARIB, NIRAPARIB (trade name ZEJULA), among others. Typically, therapies include at least one immunotherapy (or an immunotherapeutic agent). Immunotherapy refers generally to methods of enhancing an immune response against a given cancer type. In certain embodiments, immunotherapy refers to methods of enhancing a T cell response against a tumor or cancer.

In some embodiments, the immunotherapy or immunotherapeutic agent targets an immune checkpoint molecule. Certain tumors are able to evade the immune system by co-opting an immune checkpoint pathway. Thus, targeting immune checkpoints has emerged as an effective approach for countering a tumor's ability to evade the immune system and activating anti-tumor immunity against certain cancers. Pardoll, Nature Reviews Cancer, 2012, 12:252-264.

In certain embodiments, the immune checkpoint molecule is an inhibitory molecule that reduces a signal involved in the T cell response to antigen. For example, CTLA4 is expressed on T cells and plays a role in downregulating T cell activation by binding to CD80 (aka B7.1) or CD86 (aka B7.2) on antigen presenting cells. PD-1 is another inhibitory checkpoint molecule that is expressed on T cells. PD-1 limits the activity of T cells in peripheral tissues during an inflammatory response. In addition, the ligand for PD-1 (PD-L1 or PD-L2) is commonly upregulated on the surface of many different tumors, resulting in the downregulation of anti-tumor immune responses in the tumor microenvironment. In certain embodiments, the inhibitory immune checkpoint molecule is CTLA4 or PD-1. In other embodiments, the inhibitory immune checkpoint molecule is a ligand for PD-1, such as PD-L1 or PD-L2. In other embodiments, the inhibitory immune checkpoint molecule is a ligand for CTLA4, such as CD80 or CD86. In other embodiments, the inhibitory immune checkpoint molecule is lymphocyte activation gene 3 (LAG3), killer cell immunoglobulin like receptor (KIR), T cell membrane protein 3 (TIM3), galectin 9 (GAL9), or adenosine A2a receptor (A2aR).

Antagonists that target these immune checkpoint molecules can be used to enhance antigen-specific T cell responses against certain cancers. Accordingly, in certain embodiments, the immunotherapy or immunotherapeutic agent is an antagonist of an inhibitory immune checkpoint molecule. In certain embodiments, the inhibitory immune checkpoint molecule is PD-1. In certain embodiments, the inhibitory immune checkpoint molecule is PD-L1. In certain embodiments, the antagonist of the inhibitory immune checkpoint molecule is an antibody (e.g., a monoclonal antibody). In certain embodiments, the antibody or monoclonal antibody is an anti-CTLA4, anti-PD-1, anti-PD-L1, or anti-PD-L2 antibody. In certain embodiments, the antibody is a monoclonal anti-PD-1 antibody. In some embodiments, the antibody is a monoclonal anti-PD-L1 antibody. In certain embodiments, the monoclonal antibody is a combination of an anti-CTLA4 antibody and an anti-PD-1 antibody, an anti-CTLA4 antibody and an anti-PD-L1antibody, or an anti-PD-L1 antibody and an anti-PD-1 antibody. In certain embodiments, the anti-PD-1 antibody is one or more of pembrolizumab (Keytruda®) or nivolumab (Opdivo®). In certain embodiments, the anti-CTLA4 antibody is ipilimumab (Yervoy®). In certain embodiments, the anti-PD-L1 antibody is one or more of atezolizumab (Tecentriq®), avelumab (Bavencio®), or durvalumab (Imfinzi®).

In certain embodiments, the immunotherapy or immunotherapeutic agent is an antagonist (e.g. antibody) against CD80, CD86, LAG3, KIR, TIM3, GAL9, or A2aR. In other embodiments, the antagonist is a soluble version of the inhibitory immune checkpoint molecule, such as a soluble fusion protein comprising the extracellular domain of the inhibitory immune checkpoint molecule and an Fc domain of an antibody. In certain embodiments, the soluble fusion protein comprises the extracellular domain of CTLA4, PD-1, PD-L1, or PD-L2. In some embodiments, the soluble fusion protein comprises the extracellular domain of CD80, CD86, LAG3, KIR, TIM3, GAL9, or A2aR. In one embodiment, the soluble fusion protein comprises the extracellular domain of PD-L2 or LAG3.

In certain embodiments, the immune checkpoint molecule is a co-stimulatory molecule that amplifies a signal involved in a T cell response to an antigen. For example, CD28 is a co-stimulatory receptor expressed on T cells. When a T cell binds to antigen through its T cell receptor, CD28 binds to CD80 (aka B7.1) or CD86 (aka B7.2) on antigen-presenting cells to amplify T cell receptor signaling and promote T cell activation. Because CD28 binds to the same ligands (CD80 and CD86) as CTLA4, CTLA4 is able to counteract or regulate the co-stimulatory signaling mediated by CD28. In certain embodiments, the immune checkpoint molecule is a co-stimulatory molecule selected from CD28, inducible T cell co-stimulator (ICOS), CD137, OX40, or CD27. In other embodiments, the immune checkpoint molecule is a ligand of a co-stimulatory molecule, including, for example, CD80, CD86, B7RP1, B7-H3, B7-H4, CD137L, OX40L, or CD70.

Agonists that target these co-stimulatory checkpoint molecules can be used to enhance antigen-specific T cell responses against certain cancers. Accordingly, in certain embodiments, the immunotherapy or immunotherapeutic agent is an agonist of a co-stimulatory checkpoint molecule. In certain embodiments, the agonist of the co-stimulatory checkpoint molecule is an agonist antibody and preferably is a monoclonal antibody. In certain embodiments, the agonist antibody or monoclonal antibody is an anti-CD28 antibody. In other embodiments, the agonist antibody or monoclonal antibody is an anti-ICOS, anti-CD137, anti-OX40, or anti-CD27 antibody. In other embodiments, the agonist antibody or monoclonal antibody is an anti-CD80, anti-CD86, anti-B7RP1, anti-B7-H3, anti-B7-H4, anti-CD137L, anti-OX40L, or anti-CD70 antibody.

In certain embodiments, the status of a nucleic acid variant from a sample from a subject as being of somatic or germline origin may be compared with a database of comparator results from a reference population to identify customized or targeted therapies for that subject. Typically, the reference population includes patients with the same cancer or disease type as the subject and/or patients who are receiving, or who have received, the same therapy as the subject. A customized or targeted therapy (or therapies) may be identified when the nucleic variant and the comparator results satisfy certain classification criteria (e.g., are a substantial or an approximate match).

In certain embodiments, the customized therapies described herein are typically administered parenterally (e.g., intravenously or subcutaneously). Pharmaceutical compositions containing an immunotherapeutic agent are typically administered intravenously. Certain therapeutic agents are administered orally. However, customized therapies (e.g., immunotherapeutic agents, etc.) may also be administered by any method known in the art, for example, buccal, sublingual, rectal, vaginal, intraurethral, topical, intraocular, intranasal, and/or intraauricular, which administration may include tablets, capsules, granules, aqueous suspensions, gels, sprays, suppositories, salves, ointments, or the like.

In some embodiments, therapy is customized based on the status of a nucleic acid variant as being of somatic or germline origin. In some embodiments, determination of the levels of particular cell types, e.g., immune cell types, including rare immune cell types, facilitates selection of appropriate treatment.

The present methods can be used to diagnose the presence of a condition, e.g., cancer or precancer, in a subject, to characterize a condition (such as to determine a cancer stage or heterogeneity of a cancer), to monitor a subject's response to receiving a treatment for a condition (such as a response to a chemotherapeutic or immunotherapeutic), assess prognosis of a subject (such as to predict a survival outcome in a subject having a cancer), to determine a subject's risk of developing a condition, to predict a subsequent course of a condition in a subject, to determine metastasis or recurrence of a cancer in a subject (or a risk of cancer metastasis or recurrence), and/or to monitor a subject's health as part of a preventative health monitoring program (such as to determine whether and/or when a subject is in need of further diagnostic screening). The methods according to the present disclosure can also be useful in predicting a subject's response to a particular treatment option. Successful treatment options may increase the amount of copy number variation, rare mutations, and/or cancer-related epigenetic signatures (such as hypermethylated regions or hypomethylated regions) detected in a subject's blood (such as in DNA isolated from a buffy coat sample or any other sample comprising cells, such as a blood sample (e.g., a whole blood sample, a leukapheresis sample, or a PBMC sample) from the subject) if the treatment is successful as more cancer cells may die and shed DNA, or if a successful treatment results in an increase or decrease in the quantity of a specific immune cell type in the blood and an unsuccessful treatment results in no change. In other examples, this may not occur. In another example, certain treatment options may be correlated with genetic profiles of cancers over time. This correlation may be useful in selecting a therapy for a subject. In some embodiments, determination of the metastasis site facilitates selection of appropriate treatment.

In some embodiments, therapy is customized based on the status of a detected nucleic acid variant as being of somatic or germline origin. In some embodiments, essentially any cancer therapy (e.g., surgical therapy, radiation therapy, chemotherapy, and/or the like) may be included as part of these methods. Typically, customized therapies include at least one immunotherapy (or an immunotherapeutic agent). Immunotherapy refers generally to methods of enhancing an immune response against a given cancer type. In certain embodiments, immunotherapy refers to methods of enhancing a T cell response against a tumor or cancer.

In certain embodiments, the status of a nucleic acid variant from a sample from a subject as being of somatic or germline origin may be compared with a database of comparator results from a reference population to identify customized or targeted therapies for that subject. Typically, the reference population includes patients with the same cancer or disease type as the subject and/or patients who are receiving, or who have received, the same therapy as the subject. A customized or targeted therapy (or therapies) may be identified when the nucleic variant and the comparator results satisfy certain classification criteria (e.g., are a substantial or an approximate match).

The disclosed methods can include evaluating (such as quantifying) and/or interpreting at least one cell material released from a potential metastasis site (such as at least one cell material in a sample from a subject) and/or cell types that contribute to DNA, such as cfDNA, in one or more samples collected from a subject at one or more timepoints in comparison to a selected baseline value or reference standard (or a selected set of baseline values or reference standards). A baseline value or reference standard may be a presence or level of at least one cell material and/or a quantity of cell types measured in one or more samples (such as an average quantity or range of quantities of cell types present in at least two samples) collected from the subject at one or more time points, such as prior to receiving a treatment, prior to diagnosis of a condition (such as a cancer), or as part of a preventative health monitoring program. A baseline value or reference standard may be a presence or level of at least one cell material and/or a quantity of cell types measured with respect to one or more samples (such as an average quantity or range of quantities of cell types present in at least two samples) collected at one or more timepoints from one or more subjects that do not have the condition (such as a healthy subject that does not have a cancer), one or more subjects that responded favorably to the treatment, or one or more subjects that have not received the treatment. In certain embodiments, the baseline value or reference standard utilized is a standard or profile derived from a single reference subject. In other embodiments, the baseline value or reference standard utilized is a standard or profile derived from averaged data from multiple reference subjects. The reference standard, in various embodiments, can be a single value, a mean, an average, a numerical mean or range of numerical means, a numerical pattern, or a graphical pattern created from the cell type quantity data derived from a single reference subject or from multiple reference subjects. Selection of the particular baseline values or reference standards, or selection of the one or more reference subjects, depends upon the use to which the methods described herein are to be put by, for example, a research scientist or a clinician (such as a physician).

In some embodiments, methods are provided for monitoring a response (such as a change in disease state, such as a presence or absence of a metastasis in a subject, such as measured by assessing a presence or level of at least one cell material released from a potential metastasis site in a sample from the subject) of a subject to a treatment (such as a chemotherapy or an immunotherapy). In certain embodiments, one or more samples is collected from the subject at at least 1-10, at least 1-5, at least 2-5, or at least 1, at least 2, least 3, at least 4, at least 5, at least 6, at least 7, at least 8, at least 9, at least 10, at least 15, or at least 20 time points prior to the subject receiving the treatment. In certain embodiments, one or more samples is collected from the subject at at least 1-10, at least 1-5, at least 2-5, or at least 1, at least 2, least 3, at least 4, at least 5, at least 6, at least 7, at least 8, at least 9, at least 10, at least 15, or at least 20 time points after the subject has received the treatment. Sample collection from a subject can be ongoing during and/or after treatment to monitor the subject's response to the treatment.

In some embodiments, samples are not collected from a subject prior to diagnosis of a condition (such as a cancer) or prior to receiving a treatment. In such embodiments, wherein the response of a subject to a treatment or the course or stage of a condition (such as a cancer) in the subject is being monitored over time, cell types are compared between samples taken at at least 2-10, at least 2-5, at least 3-6, or at least 2, such as at least 3, at least 4, at least 5, at least 6, at least 7, at least 8, at least 9, at least 10, at least 15, or at least 20 time points collected after the subject has been diagnosed and/or after the subject has received the treatment. Sample collection from a subject can be ongoing during and/or after treatment to monitor the subject's response to the treatment.

In some embodiments of the disclosed methods, one or more samples is collected from a subject at least once per year, such as about 1-12 times or about 2-6 times, such as about 1, 2, 3, 4, 5, 6, 7, 8, 9, 10, 11, or 12 times per year. In other embodiments, one or more samples is collected from the subject less than once per year, such as about once every 13, 14, 15, 16, 17, 18, 19, 20, 21, 22, 23, or 24 months. In some embodiments, one or more samples is collected from the subject about once every 1-5 years or about once every 1-2 years, such as about every 1, 1.5, 2, 2.5, 3, 3.5, 4, 4.5, or 5 years.

In other embodiments of the disclosed methods, one or more samples are collected from a subject at least once per week, such as on 1-4 days, 1-2 days, or on 1, 2, 3, 4, 5, 6, or 7 days per week. In certain embodiments, one or more samples is collected from the subject at least once per month, such as 1-15 times, 1-10 times, 2-5 times, or 1, 2, 3, 4, 5, 6, 7, 8, 9, 10, 11, 12, 13, 14, or 15 times per month. In other embodiments, one or more samples is collected from the subject every month, every 2 months, every 3 months, every 4 months, every 5 months, every 6 months, every 7 months, every 8 months, every 9 months, every 10 months, every 11 months, or every 12 months. In some embodiments, one or more samples is collected from the subject at least once per day, such as 1, 2, 3, 4, 5, or 6 times per day. Selection of the one or more sample collection timepoints (e.g., the frequency of sample collection), or of the number of samples to be collected at each timepoint, depends upon the use to which the methods described herein are to be put by, for example, a research scientist or a clinician (such as a physician).

In certain embodiments, the customized therapies described herein are typically administered parenterally (e.g., intravenously or subcutaneously). Pharmaceutical compositions containing an immunotherapeutic agent are typically administered intravenously. Certain therapeutic agents are administered orally. However, customized therapies (e.g., immunotherapeutic agents, etc.) may also be administered by methods such as, for example, buccal, sublingual, rectal, vaginal, intraurethral, topical, intraocular, intranasal, and/or intraauricular, which administration may include tablets, capsules, granules, aqueous suspensions, gels, sprays, suppositories, salves, ointments, or the like.

### IV. Kits

Also provided are kits comprising the compositions as described herein. The kits can be useful in performing the methods as described herein. In some embodiments, a kit comprises reagents for capturing tissue-specific cell materials. In some embodiments, a kit comprises agents or other reagents for partitioning each sample into a plurality of subsamples as described herein. In some embodiments, the agent for partitioning each sample is an antibody is specific for methyl cytosine in DNA. The kit may comprise additional elements as discussed herein. In some embodiments, a kit comprises instructions for performing a method described herein.

Kits may further comprise a plurality of oligonucleotide probes that selectively hybridize to least 5, 6, 7, 8, 9, 10, 20, 30, 40 or all genes selected from the group consisting of ALK, APC, BRAF, CDKN2A, EGFR, ERBB2, FBXW7, KRAS, MYC, NOTCH1, NRAS, PIK3CA, PTEN, RBI, TP53, MET, AR, ABLl, AKTl, ATM, CDHl, CSFIR, CTNNBl, ERBB4, EZH2, FGFRl, FGFR2, FGFR3, FLT3, GNA11, GNAQ, GNAS, HNF1A, HRAS, IDH1, IDH2, JAK2, JAK3, KDR, KIT, MLH1, MPL, NPM1, PDGFRA, PROC, PTPN11, RET,SMAD4, SMARCB1, SMO, SRC, STK11, VHL, TERT, CCND1, CDK4, CDKN2B, RAF1, BRCA1, CCND2, CDK6, NF1, TP53, ARID 1 A, BRCA2, CCNE1, ESR1, RIT1, GATA3, MAP2K1, RHEB, ROS1, ARAF, MAP2K2, NFE2L2, RHOA, and NTRKl. The number genes to which the oligonucleotide probes can selectively hybridize can vary. For example, the number of genes can comprise 1, 2,3, 4, 5, 6, 7, 8, 9, 10, 11, 12, 13, 14, 15, 16, 17, 18, 19, 20, 21, 22, 23, 24, 25, 26, 27, 28, 29, 30,31, 32, 33, 34, 35, 36, 37, 38, 39, 40, 41, 42, 43, 44, 45, 46, 47, 48, 49, 50, 51, 52, 53, or 54. The kit can include a container that includes the plurality of oligonucleotide probes and instructions for performing any of the methods described herein.

The oligonucleotide probes can selectively hybridize to exon regions of the genes, e.g., of the at least 5 genes. In some cases, the oligonucleotide probes can selectively hybridize to at least 30 exons of the genes, e.g., of the at least 5 genes. In some cases, the multiple probes can selectively hybridize to each of the at least 30 exons. The probes that hybridize to each exon can have sequences that overlap with at least 1 other probe. In some embodiments, the oligoprobes can selectively hybridize to non-coding regions of genes disclosed herein, for example, intronic regions of the genes. The oligoprobes can also selectively hybridize to regions of genes comprising both exonic and intronic regions of the genes disclosed herein.

Any number of exons can be targeted by the oligonucleotide probes. For example, at least 1, 2, 3, 4, 5, 6, 7, 8, 9, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290,, 295, 300, 400, 500, 600, 700, 800, 900, 1,000, or more, exons can be targeted.

The kit can comprise at least 4, 5, 6, 7, or 8 different library adapters having distinct molecular barcodes and identical sample barcodes. The library adapters may not be sequencing adapters. For example, the library adapters do not include flow cell sequences or sequences that permit the formation of hairpin loops for sequencing. The different variations and combinations of molecular barcodes and sample barcodes are described throughout, and are applicable to the kit. Further, in some cases, the adapters are not sequencing adapters. Additionally, the adapters provided with the kit can also comprise sequencing adapters. A sequencing adapter can comprise a sequence hybridizing to one or more sequencing primers. A sequencing adapter can further comprise a sequence hybridizing to a solid support, e.g., a flow cell sequence. For example, a sequencing adapter can be a flow cell adapter. The sequencing adapters can be attached to one or both ends of a polynucleotide fragment. In some cases, the kit can comprise at least 8 different library adapters having distinct molecular barcodes and identical sample barcodes. The library adapters may not be sequencing adapters. The kit can further include a sequencing adapter having a first sequence that selectively hybridizes to the library adapters and a second sequence that selectively hybridizes to a flow cell sequence. In another example, a sequencing adapter can be hairpin shaped. For example, the hairpin shaped adapter can comprise a complementary double stranded portion and a loop portion, where the double stranded portion can be attached {e.g., ligated) to a double-stranded polynucleotide. Hairpin shaped sequencing adapters can be attached to both ends of a polynucleotide fragment to generate a circular molecule, which can be sequenced multiple times. A sequencing adapter can be up to 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more bases from end to end. The sequencing adapter can comprise 20-30, 20-40, 30-50, 30-60, 40-60, 40-70, 50-60, 50-70, bases from end to end. In a particular example, the sequencing adapter can comprise 20-30 bases from end to end. In another example, the sequencing adapter can comprise 50-60 bases from end to end. A sequencing adapter can comprise one or more barcodes. For example, a sequencing adapter can comprise a sample barcode. The sample barcode can comprise a pre-determined sequence. The sample barcodes can be used to identify the source of the polynucleotides. The sample barcode can be at least 1, 2, 3, 4, 5, 6, 7, 8, 9, 10, 11, 12, 13, 14, 15, 16, 17, 18, 19, 20, 21, 22, 23, 24, 25, or more (or any length as described throughout) nucleic acid bases, e.g., at least 8 bases. The barcode can be contiguous or non-contiguous sequences, as described herein.

The library adapters can be blunt ended and Y-shaped and can be less than or equal to 40 nucleic acid bases in length. Other variations of the can be found throughout and are applicable to the kit.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the disclosure described herein may be employed in practicing the invention. It is therefore contemplated that the disclosure shall also cover any such alternatives, modifications, variations or equivalents. It is intended that the following claims define the scope of the invention and that methods and structures within the scope of these claims and their equivalents be covered thereby. While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, systems, computer readable media, and/or component features, steps, elements, or other aspects thereof can be used in various combinations.

All patents, patent applications, websites, other publications or documents, accession numbers and the like cited herein are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number, if applicable. Likewise, if different versions of a publication, website or the like are published at different times, the version most recently published at the effective filing date of the application is meant, unless otherwise indicated.

### EXAMPLES

### Example 1: Analysis of a Sample to Detect the Presence or Level of Cell Material Released From a Potential Metastasis Site in a Subject

A set of patient samples from subjects having a cancer, tumor, or neoplasm are analyzed by a blood-based NGS assay to detect the presence or absence of metastasis. cfDNA is extracted from the samples of these patients and subjected to Tet-assisted bisulfite (TAB) conversion. β-glucosyl transferase is first used to protect 5hmC (forming 5-glucosylhydroxymethylcytosine (5ghmC)), then a TET protein, such as mTet1, is used to convert 5mC to 5caC. Bisulfite treatment is then used to convert unmodified cytosine and 5caC to U, while 5ghmC remains unaffected. The DNA molecules are cleaned and concentrated in preparation for the enzymatic steps of library preparation.

After concentrating the DNA, first adapters are added to the DNA by ligation to the 3′ ends thereof. The adapter is used as a priming site for second-strand synthesis using a universal primer and a DNA polymerase. The first adapter comprises a biotin, and nucleic acid ligated to the first adapter is bound to beads comprising streptavidin. A second adapter is then ligated to the 3′ end of the second strand of the now double-stranded molecules. These adapters contain non-unique molecular barcodes. After ligation, the DNA is amplified by PCR. Following PCR, amplified DNA is washed and concentrated prior to capture of

fragmentation variable target regions. Once concentrated, the amplified DNA is combined with a salt buffer and biotinylated RNA probes that comprise probes for tissue-specific regions of interest, including probes for a fragmentation variable target region set and this mixture is incubated overnight. The probes for the fragmentation target region set comprise oligonucleotides targeting a selection of tissue-specific cfDNA fragmentation patterns.

The biotinylated RNA probes (hybridized to DNA) are captured by streptavidin magnetic beads and separated from the amplified DNA that are not captured by a series of salt based washes, thereby enriching the sample. After enrichment, an aliquot of the enriched sample is sequenced using Illumina NovaSeq sequencer. The sequence reads generated by the sequencer are then analyzed using bioinformatic tools/algorithms. The molecular barcodes are used to identify unique molecules. The method described in this example can provide information about disease state, including whether metastasis is present. The fragmentation variable target region sequences are analyzed to detect cfDNA molecules in regions that have been shown to be differentially fragmented in a tissue-specific manner in healthy tissues.

Optionally, sequence-variable target region sequences may also be captured from the sample and analyzed by detecting genomic alterations such as SNVs, insertions, deletions and fusions that can be called with enough support that differentiates real tumor variants from technical errors (for e.g., PCR errors, sequencing errors). Such an analysis can be used to determine occurrence or recurrence of a primary cancer, tumor, or neoplasm. The results of both analyses are combined.

The method produces a final metastasis present/absent call and/or information about a primary cancer, tumor, or neoplasm based on the extent to which abnormalities (e.g., deviations from a healthy profile) are detected in the levels, identities, and locations of the cfDNA, optionally in combination with genomic alterations detected in the sequence-variable target regions and/or other signals from epigenetic target regions such as DNA methylation.

### Example 2: Analysis of a Sample to Detect Whether the Presence or Level of Cell Material Released From a Potential Metastasis Site in a Subject Has Changed Relative to the Presence or Level of the Cell Material in a Previous Sample From the Subject

A set of patient samples from subjects from which a set of previous samples were obtained are analyzed by a blood-based NGS assay to detect the presence or absence of metastasis. cfDNA is extracted from the samples and partitioned based on cytosine methylation levels. The cfDNA is contacted with an antibody that recognizes methyl cytosine, then immunoprecipitated using magnetic beads conjugated to protein G, thus partitioning hypermethylated DNA from hypomethylated DNA. Any non-methylated or less methylated DNA is washed away from the beads with buffers containing increasing concentrations of salt. Finally, a high salt buffer is used to wash the heavily methylated DNA away from the antibody to provide a hypermethylated partition, an intermediate partition, and a hypomethylated partition.

After concentrating the cfDNA in the partitions, first adapters are added to the cfDNA by ligation to the 3′ ends thereof. The adapter is used as a priming site for second-strand synthesis using a universal primer and a DNA polymerase. The first adapter comprises a biotin, and nucleic acid ligated to the first adapter is bound to beads comprising streptavidin. A second adapter is then be ligated to the 3′ end of the second strand of the now double-stranded molecules. These adapters contain non-unique molecular barcodes, and each partition is ligated with adapters having non-unique molecular barcodes that is distinguishable from the barcodes in the adapters used in the other partitions. After ligation, the partitions are pooled together and are amplified by PCR.

Following PCR, amplified DNA is washed and concentrated prior to enrichment. Once concentrated, the amplified DNA is combined with a salt buffer and biotinylated RNA probes that comprise probes for an epigenetic target region set and optionally, probes for a sequence-variable target region set, and this mixture is incubated overnight. If used, the probes for the sequence-variable region set have a footprint of about 50 kb. The probes for the epigenetic target region set has a footprint of about 500 kb. If used, the probes for the sequence-variable target region set comprise oligonucleotides targeting at least a subset of genes described herein. The probes for the epigenetic target region set comprise oligonucleotides targeting a selection of tissue-specific hypermethylation variable target regions, tissue-specific hypomethylation variable target regions, and optionally one or more of CTCF binding target regions, transcription start site target regions, focal amplification target regions and methylation control regions.

The biotinylated RNA probes (hybridized to DNA) are captured by streptavidin magnetic beads and separated from the amplified DNA that are not captured by a series of salt-based washes, thereby enriching the sample. After enrichment, an aliquot of the enriched sample is sequenced using Illumina NovaSeq sequencer. The sequence reads generated by the sequencer are then analyzed using bioinformatic tools/algorithms. The molecular barcodes are used to identify unique molecules as well as for deconvolution of the sample into molecules that were differentially partitioned. The method described in this example, apart from providing information on the overall level of methylation (i.e., methylated cytosine residues) of a molecule based on its partition, can also provide a higher resolution information about the identity and/or location of the type of methylated cytosine. If captured, the sequence-variable target region sequences are analyzed by detecting genomic alterations such as SNVs, insertions, deletions and fusions that can be called with enough support that differentiates real tumor variants from technical errors (for e.g., PCR errors, sequencing errors). The epigenetic target region sequences are analyzed independently to detect the presence or levels of methylated cfDNA molecules in regions that have been shown to be differentially methylated in a tissue-specific manner and/or in cancer compared to normal cells. The presence or levels of methylated cfDNA are compared to the levels obtained from the previous samples from the subjects analyzed according to the method of this Example or similar methods, such as similar methods described herein. Detection of the presence or absence of a metastasis is then completed based at least in part on a determination of whether the levels have changed relative to those of the previous samples and the direction and extent of such a change. The method may also produce information about a primary cancer, tumor, or neoplasm, including recurrence status, based on the extent to which abnormalities (e.g., deviations from a healthy profile) are detected in the levels, identities, and locations of the cfDNA, optionally in combination with genomic alterations detected in the sequence-variable target regions and/or other signals from epigenetic target regions such as DNA methylation.

### Example 3: Analysis of a Sample to Detect the Level of Cell Material Released From a Potential Metastasis Site in a Subject Relative to the Level of a Comparator Cell Material

A set of patient samples are analyzed by a blood-based NGS assay to detect the presence or absence of metastasis. cfDNA is extracted from the samples, partitioned, ligated, amplified, combined with biotinylated RNA probes, enriched, and sequenced, as described in Example 2. The sequence reads generated by the sequencer are then analyzed similarly to the analysis described in Example 2. Plus, the levels of tissue-specific methylated cfDNA are compared to reference levels of tissue-specific methylated cfDNA corresponding to the same tissues. The reference levels are levels detected in samples obtained from healthy subjects and analyzed according to the method of this Example or similar methods, such as similar methods described herein. Detection of the presence or absence of a metastasis is then completed based at least in part on the direction and magnitude of the relative level of methylated cfDNA in each subject. The method may also produce information about a primary cancer, tumor, or neoplasm, including recurrence status, based on the extent to which abnormalities (e.g., deviations from a healthy profile) are detected in the levels, identities, and locations of the cfDNA, optionally in combination with genomic alterations detected in the sequence-variable target regions and/or other signals from epigenetic target regions such as DNA methylation.

1. A method of detecting the presence or absence of a metastasis in a subject, the method comprising:
detecting a presence or level of at least one cell material released from a potential metastasis site in a sample from the subject; and
detecting the presence or absence of the metastasis based at least in part on the presence or level of the at least one cell material released from the potential metastasis site;
wherein the at least one cell material released from the potential metastasis site comprises one or more of tissue-specific hydroxymethylated DNA; tissue-specific fragmented DNA; a tissue-specific modified histone; a tissue-specific bacterial nucleic acid; a tissue-specific protein or cell debris; a tissue-specific extracellular vesicle; or a tissue-specific RNA.

2. A method of detecting the presence or absence of a metastasis in a subject, the method comprising:
detecting a presence or level of at least one cell material released from a potential metastasis site in a sample from the subject;
detecting whether the presence or level of the at least one cell material released from the potential metastasis site has changed relative to the presence or level of the at least one cell material in a previous sample from the subject, wherein the previous sample was obtained at an earlier time than the sample; and
detecting the presence or absence of the metastasis based at least in part on a change, or lack thereof, in the presence or level of the at least one cell material released from the potential metastasis site between the previous sample and the sample.

3. The method of claim 2, wherein the previous sample was obtained before the subject underwent a cancer treatment, and the sample was obtained after the treatment; or the previous sample was obtained within one month of the subject receiving a cancer diagnosis; or the previous sample was obtained at least 3, 6, 9, 12, 18, or 24 months before the sample.

4. A method of detecting the presence or absence of a metastasis in a subject, the method comprising:
detecting a relative level of at least one cell material released from a potential metastasis site in a sample from the subject; and
detecting the presence or absence of the metastasis based at least in part on the relative level of the at least one cell material released from the potential metastasis site; wherein the relative level of the at least one cell material is the level of the at least one cell material relative to the level of a comparator cell material.

5. The method of claim 2, wherein the presence or level of the at least one cell material released from a potential metastasis site is a relative level, wherein the relative level of the at least one cell material is the level of the at least one cell material relative to the level of a comparator cell material, and wherein the detecting the presence or absence of the metastasis based at least in part on a change, or lack thereof, in the relative level of the at least one cell material released from the potential metastasis site between the previous sample and the sample.

6. The method of claim 4, wherein the comparator cell material comprises cell material released from one or more cell or tissue types selected from erythroid cell or tissue, healthy cell or tissue, and primary cancer, tumor, or neoplastic cell.

7. The method of claim 4, wherein the comparator cell material comprises a heterologous comparator cell material.

8. The method of claim 7, wherein the heterologous comparator cell material is cell material obtained from a reference population of subjects.

9. The method of claim 4, wherein the level of the comparator cell material is determined from samples obtained from a reference population of healthy subjects.

10. The method of claim 4, wherein the comparator cell material comprises an autologous comparator cell material.

11. The method of claim 4, wherein the autologous cell material is cell material obtained from the subject when the subject did not have a metastasis or did not have a primary cancer, tumor, or neoplasm.

12. The method of claim 4, wherein the level of the comparator cell material is determined from one or more samples obtained from the subject when the subject did not have a metastasis or did not have a primary cancer, tumor, or neoplasm.

13. The method of claim 2, wherein (a) the at least one cell material released from the potential metastasis site comprises tissue-specific methylated DNA; and/or (b) the at least one cell material released from the potential metastasis site comprises one or more of tissue-specific hydroxymethylated DNA; tissue-specific fragmented DNA; a tissue-specific modified histone; a tissue-specific bacterial nucleic acid; a tissue-specific protein or cell debris; or a tissue-specific RNA.

14. (canceled)

15. The method of claim 1, wherein the at least one cell material released from the potential metastasis site comprises:
(a) cell-free cell material;
(b) a plurality of cell materials released from the potential metastasis site;
(c) a tissue-specific bacterial nucleic acid;
(d) a tissue-specific epigenetic target region of a nucleic acid;
(e) a tissue-specific RNA selected from a tissue-specific microRNA, a tissue-specific exosomal RNA, or a tissue-specific extracellular RNA; and/or
(f) tissue-specific cell debris comprising a protein, carbohydrate, and/or cell debris marker.

16. (canceled)

17. (canceled)

18. The method of claim 15, wherein the tissue-specific bacterial nucleic acid is:
(a) released from bacteria located in the gut, mouth, or reproductive organs;
(b) released from Bilophila wadsworthia, Streptococcus bovis, Helicobacter pylori, Bacteroides fragilis, or Clostridium septicum;
(c) a cell-free nucleic acid; and/or
(d) a 16S rRNA or DNA encoding a 16S rRNA.

19.-22. (canceled)

23. The method of claim 15, wherein the tissue-specific epigenetic target region comprises a region of tissue-specific methylated DNA.

24. The method of claim 19, wherein the tissue-specific methylated DNA:
(a) comprises cfDNA; and/or
(b) is specific to the brain, lung, skin, nose, throat, liver, bone, bone marrow, pancreas. lymph nodes, bowel, rectum, colon, prostate, thyroid, bladder, head, neck, kidney, mouth, stomach, or spleen.

25.-28. (canceled)

29. The method of claim 15, wherein the tissue-specific cell debris comprises PD-L1, CTLA4, NYESO1, mesothelin, CA15-3, CA19-9, CA-125, or CA-172-4.

30.-33. (canceled)

34. The method of claim 1, wherein the potential metastasis site is the brain, lung, skin, nose, throat, liver, bone, bone marrow, pancreas, lymph nodes, bowel, rectum, colon, prostate, thyroid, bladder, head, neck, kidney, mouth, stomach, or spleen.

35. (canceled)

36. The method of claim 1, wherein the presence of the metastasis is detected based at least in part on detection of a level of at least one cell material released from the metastasis site that is higher than expected for healthy tissue that has not been invaded by a metastasis.

37. The method of claim 36, wherein the higher than expected level of at least one cell material released from the metastasis site is higher relative to the level detected in a previous sample or relative to a comparator cell material.

38. (canceled)

39. (canceled)

40. The method of claim 1, wherein the sample is a blood sample, and/or the sample comprises cfDNA.

41.-46. (canceled)

47. The method of claim 1, further comprising detecting the primary cancer, tumor, or neoplasm based at least in part on detecting at least one cell material released from one or more primary cancer, tumor, or neoplasm cells or from tissue in which the primary cancer, tumor, or neoplasm cells are located, wherein the detecting comprises capturing a plurality of sets of target regions of DNA from the sample or one or more subsamples thereof, wherein the plurality of sets of target regions comprises a sequence-variable target region set and an epigenetic target region set, thereby providing captured DNA.

48. (canceled)

49. (canceled)

50. The method of claim 1, wherein the subject
(a) was diagnosed with cancer before the sample was obtained;
(b) received treatment for a cancer before the sample was obtained;
(c) is undergoing screening for cancer; and/or
(d) has a metastasis, and the method further comprises identifying the metastasis site based at least in part on the at least one cell material.

51.-53. (canceled)

54. The method of claim 50, wherein the metastasis site is the brain, lung, skin, nose, throat, liver, bone, bone marrow, pancreas, lymph nodes, bowel, rectum, colon, prostate, thyroid, bladder, head, neck, kidney, mouth, stomach, or spleen.

55.-70. (canceled)

71. The method of claim 1, wherein the detecting the presence, level, or relative level of the at least one cell material from the potential metastasis site comprises subjecting the sample or a subsample thereof to a procedure that affects a first nucleobase in DNA differently from a second nucleobase in DNA, wherein the first nucleobase is a modified or unmodified nucleobase, the second nucleobase is a modified or unmodified nucleobase different from the first nucleobase, and the first nucleobase and the second nucleobase have the same base pairing specificity.

72. (canceled)

73. The method of claim 71, wherein the first nucleobase is a modified or unmodified cytosine, and the second nucleobase is a modified or unmodified cytosine.

74.-76. (canceled)

77. The method of claim 1, wherein the detecting the presence, level, or relative level of the at least one cell material from the potential metastasis site comprises detecting nucleic acids obtained from the sample or a subsample thereof, optionally wherein the detecting comprises sequencing nucleic acids obtained from the sample or a subsample thereof.

78.-81. (canceled)

82. The method of claim 47, wherein the epigenetic target regions comprise a hypermethylation variable target region set and/or a fragmentation variable target region set.

83.-86. (canceled)
